(19) United States
(12) Patent Application Publication
(10) Pub. No.: US 2021/0353566 A1
(43) Pub. Date: Nov. 18, 2021

(54) THE USE OF CHOLINE SUPPLEMENTATION AS THERAPY FOR APOE4-RELATED DISORDERS

(71) Applicants: Massachusetts Institute of Technology, Cambridge, MA (US); Whitehead Institute for Biomedical Research, Cambridge, MA (US)

(72) Inventors: Li-Huei Tsai, Cambridge, MA (US); Yuan-Ta Lin, Medford, MA (US); Julia Bonner, Somerville, MA (US); Priyanka Narayan, Cambridge, MA (US); Grzegorz Sienski, Cambridge, MA (US)

(73) Assignees: Massachusetts Institute of Technology, Massachusetts, MA (US); Whitehead Institute for Biomedical Research, Cambridge, MA (US)

(21) Appl. No.: 17/318,921

(22) Filed: May 12, 2021

Related U.S. Application Data
(60) Provisional application No. 63/023,698, filed on May 12, 2020.

(51) Int. Cl. A61K 31/14; A61P 25/28; A61P 3/06

(52) U.S. Cl. CPC A61K 31/14; A61P 3/06; A61P 25/28

(57) ABSTRACT

The invention relates to methods of using choline supplementation for treating APOE4-related disorders. In particular the methods are accomplished by administering choline treatment paradigms to re-establish lipid homeostasis in APOE4 carriers.

### CROSS REFERENCE TO RELATED APPLICATION

This application claims the benefit under 35 U.S.C. § 119(e) of U.S. Provisional Application No. 63/023,698, filed May 12, 2020, entitled “THE USE OF CHOLINE SUPPLEMENTATION AS THERAPY FOR APOE4-RELATED DISORDERS,” the entire disclosure of which is hereby incorporated by reference in its entirety.

### FEDERALLY SPONSORED RESEARCH

This invention was made with Government support under Grant No. K99 AG055697 and AG062377 awarded by the National Institutes of Health (NIH). The Government has certain rights in the invention.

### FIELD OF THE INVENTION

The present invention relates generally to methods of using choline supplementation for treating APOE4-related disorders.

### BACKGROUND OF THE INVENTION

Apolipoprotein E 4 (APOE4) is the single strongest genetic contributor to sporadic Alzheimer's Disease (AD) (Bu 2009). Possession of a single APOE4 allele increases the risk of AD incidence 3 fold, and with two E4 alleles, 15 fold (relative to APOE3/APOE3). The APOE4 isoform has also been linked with increased levels of low density lipoprotein (LDL) and has been demonstrated to be a risk factor for several disorders associated with lipid dysregulation.

### SUMMARY OF THE INVENTION

The invention relates, in one aspect, to the discovery that the presence of the APOE4 allele creates an increased requirement for choline to maintain lipid homeostasis, which can be mitigated through long term supplementation.

Accordingly, one aspect of the present invention provides a method for treating a subject for an APOE4-related disorder comprising determining the presence or absence of an ApoE4 gene in a subject having an APOE4 related disorder and delivering to the subject an effective amount of choline supplementation if the subject has an ApoE4 gene. In some embodiments, the effective amount is an effective daily dose of greater than 550 mg.

The APOE4-related disorder to be treated in the methods described herein can be Alzheimer's Disease (AD), cardiovascular disease, atherosclerosis, traumatic brain injury (TBI), Cerebral Amyloid Angiopathy (CAA), dementia with Lewy bodies (DLB), tauopathy, cerebrovascular disease, multiple sclerosis, and vascular dementia. In some embodiments, the APOE4-related disorder further comprises APOE4-mediated lipid dysfunction. In some embodiments, the APOE4-mediated lipid dysfunction comprises an accumulation of lipid droplets in microglia and/or an accumulation of lipid droplets in astrocytes.

In some aspects, the present invention is a method of reducing APOE4-mediated lipid dysfunction in a subject comprising identifying a subject in need of reducing APOE4-mediated lipid dysfunction and administering to the subject an effective amount of choline supplementation, wherein APOE4-mediated lipid dysfunction comprises an accumulation of lipid droplets in microglia, an accumulation of lipid droplets in astrocytes, and/or an increase in inflammatory cytokine IL-1B in microglia cells following activation with interferon gamma.

In other aspects, the present invention is a method of reducing amyloid β (Aβ) deposition in a subject comprising administering to the subject an effective amount of choline supplementation for reducing amyloid β (Aβ) deposition, wherein the subject has been identified as having an ApoE4 gene and wherein the choline supplementation is administered to the subject for at least 3 months.

In some aspects, the effective amount of choline supplementation of the present invention is an effective amount for altering phosphatidylcholine (PC) metabolism in the subject. In some embodiments, altering PC metabolism in a subject comprises increased expression of one or more of the following genes Pld3, S1pr1, or Plpp3 in astrocytes and/or increased expression of one or more of genes Lpcat2, P2ry12, Tgfbr1, Gpr34, Lyn, or Picalm in microglia relative to a control.

In other aspects, the effective amount of choline supplementation of the present invention is an effective amount for normalizing microglial activation in the subject. In some embodiments, normalizing microglial activation comprises decreased expression of IL-1b induction following activation with interferon gamma relative to a control.

In yet other aspects, the effective amount of choline supplementation of the present invention is an effective amount decreasing lipid droplet accumulation in the liver of the subject.

In some aspects, the choline supplementation of the present invention comprises a choline salt. In some embodiments, the choline salt is choline chloride, choline bitartrate or choline stearate. In some embodiments, the choline supplementation is administered to a subject once a day, twice a day, or three times a day. In some embodiments, the choline supplementation is administered to a subject for at least 3 months. In some embodiments, the choline supplementation is administered to a subject for at least 6 months. In some embodiments, the choline supplementation is administered to a subject for at least 12 months.

In some aspects, the method of the present invention further comprises administering a cholinesterase inhibitor to the subject.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein. The figures are illustrative only and are not required for enablement of the invention disclosed herein.

FIGS. 1A-1F show APOE4 astrocytes exhibit lipid dysregulation. FIG. 1A is a schematic depicting the use of isogenic astrocytes derived from patient-derived iPSCs and the lipidomic analysis. FIG. 1B is a heatmap showing the fold change (log 2) between APOE4/APOE4 and APOE3/APOE3 in abundance of phospholipids (˜150 lipid species, upper panel) and triglycerides (˜120 species, lower panel). FIG. 1C is a graph presenting a fold change difference of the number of unsaturated bonds in fatty acids attached to triglycerides (TAGs). FIG. 1D is a fluorescent microscopy images of the iPSC-derived astrocytes stained with LipidTox right, quantification of the lipid droplet number per cell (each point (n) is an average of four wells, n=7). Data is represented as mean±SD (Student's t-test, **** p≤0.0001). FIG. 1E is a fluorescent microscopy images of the APOE3/APOE3 and APOE4/APOE4 astrocytes stained with an anti-Perilipin 2 antibody. Right, quantification of the Perilipin-2 foci per cell (each point (n) is an average of four wells, n=4). Data is represented as mean±SD (Student's t-test, **** p≤0.0001). FIG. 1F is a quantification of lipid droplets in astrocytes treated with vehicle (control) or with oleic acid (20 μM) (n=4 experiments). Data is represented as mean±SD (ANOVA with multiple comparisons, *** p≤0.001, **** p≤0.0001).

FIGS. 2A-2G show the APOE4-induced dysfunction is rescued by drug targeting of lipid saturation enzyme and choline supplementation. FIG. 2A shows APOE3 and APOE4 positive yeast cells relative to wildtype yeast growth on minimal CSM media, quantified as shown in FIG. 2B. FIG. 2C is a schematic of a synthetic array analysis comparing yeast knockout libraries against the growth phenotype. FIG. 2D is a plot identifying genetic nodes that modify APOE4 toxicity from the synthetic genetic array. Highlighted in red are two genes associated with fatty acid saturation status (ubx2, mga2) as well as a negative regulator of phospholipid synthesis (opi1). FIG. 2E is a schematic of Ubx2, Mga2 control of OLE1 levels. FIG. 2F shows inhibition of OLE1, the yeast homolog of the lipid desaturase SCD, significantly improves APOE4 growth. The graph shows the growth rate of APOE3 and APOE4-expressing yeast and treated with 10, 20 or 40 μM of ECC145 (OLE1 inhibitor) or vehicle (DMSO). The data was normalized to the growth of untreated yeast strain expressing GFP (control). Data are represented as mean±SD, n=3. FIG. 2G is a graph depicting growth rate of APOE4 yeast in media supplemented with ethanolamine (1 mM), choline chloride (1 mM) and choline bitartrate (100 μg/ml) (shown compared to the growth of the APOE3 strain). Data is represented as mean±SD, n=6 (Student's t-test, ns p>0.05; *** p≤0.001).

FIGS. 3A-3C show choline rescues APOE4-mediated lipid dysfunction in human iPSC-derived astrocytes. FIG. 3A shows fluorescent microscopy images of the iPSC-derived astrocytes stained with LipidTox after culture using media supplemented with vehicle or CDP-choline (100 μM). Right, quantification of the lipid droplet number per cell (n=4 experiments). Data is represented as mean±SD (ANOVA with multiple comparisons, *** p≤0.001). FIG. 3B shows intracellular TAGs extracted from APOE3 and APOE4 astrocytes grown in regular media or media supplemented with CDP-choline (100 μM). Data is represented as mean±SD, n=3 experiments (Student's t-test, ** p≤0.01). FIG. 3C shows fold change of number of unsaturated bonds in fatty acids in TAGs. Lipid were extracted from APOE3 and APOE4 iPSC-derived astrocytes grown in a regular media or the media supplemented with CDP-choline (100 μM). Data is represented as mean±SD, n=3 experiments.

FIGS. 4A-4C show APOE4 lipid dysregulation and response to chemical intervention is independent of genetic background. Following extended culturing, APOE4 astrocytes derived from an independent human show increased lipid accumulation in trends towards lipid droplet numbers as in FIG. 4A and significantly increased lipid droplet volume as in FIG. 4B compared to isogenic APOE3. Data is represented as mean±SD, n=5 (Wilcoxon-Mann-Whitney test, ** p≤0.01). FIG. 4C shows second line APOE4 astrocytes show reduced lipid droplet accumulation following chemical targeting of TAG synthesis during extended culturing (noted drug concentration was added at Day 1 of culture, lipid droplets were measured at Day 14). Data is represented as mean±SD, n=3-5 (ANOVA with multiple comparisons, p=0.01).

FIGS. 5A-5C show APOE4 microglia show aberrant lipid accumulation which may be modified by supplementation with choline FIG. 5A shows APOE4 microglia show greater numbers of lipid droplet positive cells compared to isogenic controls under standard culturing conditions. Data is represented as mean±SD, n=18 (Student's t-test, ** p≤0.0001) FIG. 5B shows APOE4 microglia have significantly increased lipid droplet volume in low choline media (15 μM Choline Chloride) conditions following extended culture and activation by interferon gamma, where no difference is detected under supplemented choline conditions (65 μM Choline Chloride). FIG. 5C shows Increased IL-1b induction in APOE4 microglia under low choline (65 μM Choline Chloride) is rescued by supplemented choline (250 μM Choline Chloride). Data is represented as mean±SD, n=3-5 (ANOVA with multiple comparisons, * p≤0.05, ** p≤0.01).

FIGS. 6A-6C show choline supplementation reduces cholesterol defects in APOE4 astrocytes: FIG. 6A shows fluorescent microscopy images of the iPSC-derived astrocytes stained with Filipin III after extended culture using media supplemented with vehicle or CDP-choline (100 μM). FIG. 6B shows quantification of the signal intensity of the Filipin III staining per cell (n=6). Data represents mean±SD (ANOVA with multiple comparisons, ** p≤0.01, *** p≤0.001). FIG. 6C shows quantification of total cholesterol detected in the media of astrocytes after extended culture in media supplemented with varying choline chloride (10, 100, 1000 μM) levels. Data is represented as mean±SD (ANOVA with multiple comparisons, ** p≤0.01, *** p≤0.001).

FIGS. 7A-7C show graphs depicting mice weight over time. FIG. 7A depicts mice fed a high choline diet (3.4 g/kg choline chloride) and minimum recommended choline diet (0.7 g/kg choline chloride) for one month. FIG. 7B depicts mice fed a sub-recommended choline diet (0.1 g/kg choline chloride) compared to standard choline (1.1 g/kg choline chloride) for one month. FIG. 7C depicts mice fed a high choline diet (3.4 g/kg choline chloride) and minimum recommended choline diet (0.7 g/kg choline chloride) for three months. In each instance, mice gained appropriate weight and showed no significant defects in body condition or appetite.

FIGS. 8A-8B show graphs depiciting lipid drop number accumulation increases in animals fed a low choline diet. FIG. 8A is a graph depicting APOE4 5×FAD (E4FAD) animals fed low choline diet (0.7 g/kg) for 3 months show trend to increased lipid droplet (LD) accumulation in the dentate gyrus (DG) region of the hippocampus, as measured by perilipin lipid droplet staining, compared to APOE3 5×FAD (E3FAD).

FIG. 8B are graphs depiciting E4FAD male mice fed a diet of high choline (3.4 g/kg) for 3 months show significantly reduced lipid droplet accumulation by perilipin-1 staining, compared to those fed low choline (0.7 g/kg) for the same length of time. Perilipin-1 intensity is shown in left panel, lipid droplet number identified by Imaris image software analysis is shown in the right panel. Data is represented as mean±SD (Wilcoxon-Mann-Whitney test, * p≤0.05).

FIGS. 9A-9F show dietary choline reduces amyloid accumulation in a human APOE4 knock-in AD mouse model in multiple regions of the hippocampus (CA1 and dentate gyrus “DG”) and using multiple amyloid antibodies (D54D2 and 12F4). FIG. 9A shows amyloid (D54D2) staining in dentate gyrus (DG) of EFAD female mice fed low choline (0.7 g/kg “MIN”) diet for three months. FIG. 9B shows amyloid (D54D2) staining in dentate gyrus (DG) of EFAD female mice fed high choline (3.4 g/kg “MAX”) diet for three months. FIG. 9C is a graph quantifying the amyloid staining levels in FIGS. 9A-9B, depicting higher amyloid accumulation in mice fed low choline compared to those fed high choline. FIG. 9D is a graph depicting CA1 amyloid accumulation is reduced in male E4FAD fed high choline (MAX) compared to low choline (MIN). FIG. 9E is a graph depicting female E4FAD mice also show reduction in an independent amyloid marker, 12F4, in the CA1 region. FIG. 9F is a graph depicting that amyloid levels were also significantly reduced in the cortices of female animals fed high choline diet compared to low choline diet as measured by Aβ40 ELISA. Data is represented as mean±SD, n=4-8 (Wilcoxon-Mann-Whitney test, * p≤0.05, *** p≤0.001).

FIG. 10 shows genes significantly upregulated in mice with high choline diet compared to minimum recommended choline diet suggest alterations in lipid pathways and reduced inflammation.

FIG. 11 shows choline supplementation rescues lipid droplet accumulation in iPS-derived astrocytes in a dose dependent manner. Increasing choline concentrations in astrocyte media (choline chloride, at 1 μM, 10 μM, and 100 μM) improves lipid droplet accumulation in a dose-dependent manner for astrocytes following extended culture (14 days).

FIGS. 12A-12C depicts RNAseq results at baseline suggesting similar mechanisms are at play in human iPSC-derived astrocytes and microglia in standard, choline limiting media. FIG. 12A shows that Stearyl co-A Desaturase (SCD), involved in fatty acid biosynthesis and unsaturating fatty acid bonds, is significantly downregulated in APOE4 astrocytes. Fatty Acid Desaturase 2 (FADS2), which also regulates unsaturation of fatty acids, is also significantly downregulated. FIG. 12B shows that under standard culturing conditions, SCD and FADS2 are also significantly downregulated in microglia, as is Diacylglycerol O-Acyltransferase 2 (DGAT2), one of two enzymes which catalyzes the final reaction in the synthesis of triglycerides (TAGs). FIG. 12C shows that following activation of microglia by interferon gamma (IFNγ), more members of the FADS gene family are significantly downregulated, and SLC44A1, Choline Transporter Like Protein 1, is now significantly upregulated. Data are depicted as mean±SD, q-value is p-value corrected for False Discovery Rate (FDR).

FIG. 13 shows RNAseq results from multiple independent cell types. Gene Ontology (GO) enrichment analysis reveals that lipid GO terms (boxed in red) are strongly enriched in astrocytes, but common to all cell types in some capacity.

FIG. 14 is a set of graphs depicting 3 months of HIGH CHOLINE (3.4 g/kg) diet reduces Aβ APOE4;5×FAD male Aβ ELISA. Cortices from male APOE4;5×FAD animals on low vs high choline diet for 3 months showed statistically reduced insoluble Aβ40 in HC, and trends towards reduction in Aβ40 CX insoluble. Data is represented as mean±SD, n=4-9 (Wilcoxon-Mann-Whitney test, * p≤0.05).

FIG. 15 is a set of graphs depicting immunohistochemistry (IHC) staining results from animals on 3 months of varying choline diet, showing trends to reduction of amyloid particles across multiple regions (cortex “CX” and dentate gyrus “DG) and antibodies. HIGH CHOLINE (3.4 g/kg) diet significantly reduces amyloid accumulation in APOE4;5×FAD females, as measured by quantification of D54D2 antibody staining of amyloid particles in the dentate gyrus (DG) (data also shown in FIG. 9B). Amyloid antibodies: 12F4, which is specific for Aβ42, including soluble, and D54D2, which recognizes total amyloid beta peptide (including Aβ40 and Aβ42) and strongly detects plaques.

FIG. 16 is a schematic depicting the extraction of mouse brains used for RNAseq. Female APOE4;5×FAD animals were treated for 3 months on HIGH CHOLINE (3.4 g/kg Choline Chloride) or LOW CHOLINE (0.7 g/kg Choline Chloride), then brains were dissected out and flash frozen. Tissue was then homogenized, stained and sorted for nuclei positive for the positive for NeuN (neurons), PU.1 (microglia), GFAP (astrocytes) and Olig2 (oligodendrocytes).

FIG. 17 is a Pathway analysis table depicting genes upregulated in astrocytes of mice on a high choline diet suggest changes to lipid regulation and reduced inflammation.

FIG. 18 is a table depicting genes upregulated and genes downregulated in astrocytes from mice on a high choline diet suggest changes to lipid regulation and reduced inflammation.

FIG. 19 is a GO Biological Process table depicting genes upregulated in microglia of mice on a high choline diet suggest changes to lipid regulation and reduced inflammation.

FIG. 20 is a table depicting genes upregulated and downregulated in microglia from mice on a high choline diet suggest changes to lipid regulation and reduced inflammation.

FIG. 21 is a set of graphs showing that low choline diets trend towards increasing lipid droplets in liver. The results indicated a modest trend to lower lipid accumulation in livers of animals on high choline. An additional trend across two trials of higher lipid burden in livers of APOE4;FAD mice on low choline diets relative to higher choline diets was inconclusive.

FIG. 22 shows modest trend to lower lipid accumulation in livers of animals on high choline diet. Trend across two trials of higher lipid burden in livers of APOE4;FAD mice on low choline diets relative to higher choline diets.

FIGS. 23A-23B shows lower choline diet applied for one month most likely does not significantly modify AD phenotypes such as amyloid accumulation. FIG. 23A is a set of graphs showing amyloid quantification in E4FAD females and E4FAD males after 1 month on a HIGH CHOLINE (3.4 g/kg) diet. FIG. 23B depicts amyloid staining in APOE4;FAD female mice.

FIG. 24 shows choline rescues rat cortical neurons expressing human APOE4. Experimental details: Over-expression of human APOE4 in rat cortical neurons causes toxicity that can be rescued by supplementing the media with choline.

### DETAILED DESCRIPTION

Prior to the present invention, the connection between two aspects of AD pathology, (1) cognitive decline and treatment with choline supplementation and (2) lipid dysregulation in APOE4 carriers, was not known. A few randomized intervention studies showed a correlation between choline supplements and improved cognitive performance in adults. However, a recent review examining a number of studies on the relationship between choline levels and neurological outcomes in adults concluded that choline supplements did not result in clear improvements in cognition in healthy adults (Leermakers E T, et al. Effects of choline on health across the life course: a systematic review. Nutr Rev 2015; 73:500-22). Additionally, a review of 12 randomized trials in 265 patients with Alzheimer's disease, concluded that there was no clear clinical benefits of lecithin supplementation for treating Alzheimer's disease (Higgins J P and Flicker L. Lecithin for dementia and cognitive impairment. Cochrane Database Syst Rev 2003:CD001015).

The present invention relates, in one aspect, to the discovery that presence of APOE4 allele creates an increased requirement for choline to maintain lipid homeostasis, which can be mitigated through long term supplementation. In some embodiments, environmental intervention with choline supplementation improves glial health and stress buffering capacity, amyloid clearance, and reduced inflammation. Increasing choline intake by choline supplementation has significant relevance to the treatment of APOE4-related disease pathologies. In some embodiments, the present invention relates to methods of using choline supplementation for treating APOE4-related disorders in a subject.

Apolipoprotein E (APOE) is a major lipoprotein in the brain that mediates trafficking and metabolism of lipids and cholesterol (Schmukler, Michaelson et al. 2018). APOE is expressed in several organs, with the highest expression in the liver, followed by the brain. Nonneuronal cells, mainly astrocytes and to some extent microglia, are the major cell types that express APOE in the brain. The APOE gene has three common alleles—APOE2, APOE3 and APOE4—which differ from each other by just two amino acids. Genome Wide Association Studies (GWAS) have identified APOE4 as the single strongest genetic contributor to sporadic Alzheimer's Disease (AD) (Bu 2009). Possession of a single APOE4 allele increases the risk of AD incidence 3 fold, and with two APOE4 alleles, 15 fold (relative to APOE3/APOE3). The APOE4 isoform has also been linked with increased levels of low density lipoprotein (LDL) and has been demonstrated to be a risk factor for cardiovascular disease and increased atherosclerosis which may have detrimental effects on brain function through decreased blood flow and altered metabolic properties (Kim, Basak et al. 2009). APOE4 is also associated with adverse outcomes after traumatic brain injury (Houlden and Greenwood 2006) and Cerebral Amyloid Angiopathy (CAA) (Rannikmae, Samarasekera et al. 2013).

Lipid metabolism is an area of active investigation in AD. A number of lipid species have been implicated in neurotoxicity or also selected as biomarkers for early diagnosis of the disease. Because the cholinergic neurons are particularly affected in AD, these data inspired a hypothesis that an increased catabolism of phospholipids limits the new membrane synthesis (Nitsch, Blusztajn et al., 1992). This is particularly important at the synapses, where vesicular signaling requires a high turnover of membranes. Because of that, therapies designed to block phospholipid breakdown by inhibiting choline esterase activity were approved in the clinic. Individuals bearing the APOE4 allele respond preferentially to the therapy (Petersen, Thomas et al., 2005, Wang, Day et al., 2014). Moreover, lipid droplet (LD) accumulation has been recently reported in both a mouse model of AD and post-mortem brains of individuals suffering from AD (Hamilton, Dufresne et al., 2015). As used herein, the term “lipid droplets” refers to a specialized cytoplasmic organelle that comprise triglycerides (TAGs), and other neutral lipids such as cholesterol esters. LDs act as a reservoir of energy for membrane biosynthesis and also protect cells from lipotoxicity by sequestering free fatty acids. Surprisingly, the present invention, at least in part, teaches that APOE4 imposes additional choline requirements resulting in a more severe cholinergic deficit than was previously appreciated in the art. As described herein, environmental interventions with choline supplementation rewire cellular metabolism to modulate the detrimental effects of APOE4 as a genetic disease risk factor. In some embodiments, choline supplementation reduces an accumulation of LDs. In some embodiments, increased availability of choline is sufficient to restore lipid homeostasis in APOE4 positive cells. In some embodiments, choline supplementation completely rescues lipid dysregulation.

As used herein, the term “APOE4-related disorder” refers to a disease or disorder associated with at least one APOE4 allele in a subject. In some embodiments, a subject with an APOE4-related disorder has one APOE4 allele. In some embodiments, a subject with an APOE4-related disorder has two APOE4 alleles. As described herein, examples of APOE4-related disorders include, but are not limited to, Alzheimer's Disease (AD), cardiovascular disease, atherosclerosis, traumatic brain injury (TBI), Cerebral Amyloid Angiopathy (CAA), dementia with Lewy bodies (DLB), tauopathy, cerebrovascular disease, multiple sclerosis, and vascular dementia. In some embodiments, the APOE4-related disorder is AD.

As described herein, an APOE4-related disorder can impact amyloid pathology. As used herein, the term “amyloid deposition” refers to a central neuropathological abnormality in APOE4-related disorders, including but not limited to, amyloid load and amyloid plaque deposition. A subject with an APOE4-related disorder may have increased amyloid load. In some embodiments, increased amyloid load effects the hippocampus of a subject with an APOE4-related disorder. In some embodiments, increased amyloid load effects the cortex of a subject with an APOE4-related disorder. In some embodiments, treating a subject with an APOE4-related disorder with choline supplementation reduces the amyloid load. In some embodiments, the reduction in amyloid load is evidenced by reduced insoluble Aβ40 levels in the cortex. In some embodiments, the reduction in amyloid load is evidenced by reduced levels of insoluble Aβ42 levels in the cortex and hippocampus. In some embodiments, treating a subject with an APOE4-related disorder with choline supplementation reduces amyloid plaque count. In some embodiments, the amyloid plaque count is reduced in the denate gyms.

In some embodiments, a subject with an APOE4-related disorder exhibits APOE4-mediated lipid dysfunction. As used here, the term “APOE4-mediated lipid dysfunction” refers to cellular phenotypes including at least, but not limited to, an accumulation of LDs in microglia, an accumulation of LD in astrocytes, microglial activation, cholesterol defects, and growth defects. One of skill in the art would appreciate that APOE4-mediated lipid dysfunction occurs at the cellular level. For example, APOE4-mediated lipid dysfunction can occur in a eukaryotic cell. In some embodiments, the eukaryotic cell is a yeast cell. As described herein, genetic nodes that modify APOE4 toxicity in a yeast cell include but are not limited to Ubx2, Mga2, and OLE1. In some embodiments, the eukaryotic cell is a non-human mammalian cell. In some embodiments, the eukaryotic cell is a human cell.

A subject may be identified for the treatment disclosed herein based on the presence or absence of an APOE4 allele. A subject may be identified as having a single APOE4 allele or two APOE4 alleles. Conventional methods for genetic analysis may be used to identify whether a subject expresses an APOE4 allele.

As used herein, the term “phosphatidylcholine metabolism” refers to genes involved in phosphatidylcholine (PC) synthesis. There are several genes that are both involved in PC metabolism and have been previously associated with AD risk or disease progression. Surprisingly, in some embodiments of the present invention, administering choline supplementation to a subject results in the increased expression of genes involved in PC metabolism including at least, but not limited to Pld3, S1pr1, or Plpp3 in astrocytes. In some embodiments, administering choline supplementation to a subject results in the increased expression of genes involved in PC metabolism including at least, but not limited to Lpcat2, P2ry12, Tgfbr1, Gpr34, Lyn, or Picalm in microglia.

As used herein, the term “microglial activation” refers to an increase in inflammatory cytokine IL-1B in microglia cells following activation with interferon gamma. In some embodiments, administering choline supplementation to a subject results in a reduction in microglial activation. In some embodiments, reduced levels of IL-1B correlate with reduced inflammation in a subject.

As used herein, the term “cholesterol defects” refers to, at least but not limited to, increased cholesterol content in a cell. In some embodiments, cholesterol defects are found in microglia and/or astrocytes of a subject with an APOE4-related disorder. In some embodiments, cholesterol defects are indicated by increased expression of Filipin III in astrocytes of a subject with an APOE4-related disorder. In some embodiments, administering choline supplementation to a subject with an APOE4-related disorder results in reduced expression of Filipin III.

As used herein, the term “choline” refers to a soluble phospholipid precursor in the synthesis of acetylcholine, phosphatidylcholine, sphingomyelin, and platelet activating factor, and is required for metabolism of triglycerides (TAGs).

As used herein, the term “choline supplementation” refers to environmental intervention by delivering and/or administering choline to a subject in need thereof. In some embodiments, choline supplementation is a dietary component or dietary additive. Choline supplementation may be delivered and/or administrated to a subject as part of a regular diet paradigm for a determined amount of time. For example, choline supplementation may be delivered and/or administered to a subject as part of a daily dietary paradigm including but not limited to once a day, twice a day, or three times a day. In some embodiments, choline supplementation is delivered and/or administered to a subject with food. In some embodiments, choline supplementation is delivered and/or administered to a subject without food. In some embodiments, choline supplementation is delivered and/or administered to a subject as part of a daily dietary routine over the course of including but not limited to, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 30 weeks, at least 40 weeks, or at least 50 weeks, at least 1 month, at least 2 months, at least 3 months, at least 4 months, at least 5 months, at least 6 months, at least 7 months at least 8 months, at least 9 months, at least 10 months, at least 11 months or at least 12 months. It can be appreciated that choline supplementation may be delivered and/or administered in the form of a choline salt. In some embodiments, the choline salt is selected from, but not limited to, a choline chloride, choline bitartrate or choline stearate.

Choline supplementation, as used herein, is delivered and/or administered to a subject in an effective amount to treat an APOE4-related disorder. As used here, the term “effective amount” refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Generally, for administration of the choline supplements an initial dosage can be greater than 500 mg/day. For the purpose of the present disclosure, a typical daily dosage might range from about any of 500 mg/day to 2,000 mg/day, 550 mg/day to 1,000 mg/day, 600 mg/day to 1,000 mg/day depending on the factors mentioned above. For repeated administrations over several days or longer, the treatment is sustained until a desired suppression of symptoms occurs or until sufficient therapeutic levels are achieved to alleviate a neurodegenerative disease, or a symptom thereof. An exemplary dosing regimen comprises administering dose of greater than about 500, 550, 600, 650, 700, 750, 800, 850, 900, 950, 1,000, 1,050, 1,100, 1,150, 1,200, 1,250, 1,300, 1,350, 1,400, 1,450, 1,500, 1, 550, 1,600, 1,650, 1,700, 1,750, 1,800, 1,850, 1,900, 1,950, or 2000 mg/day for 3 months, 6 months or a year. However, other dosage regimens may be useful, depending on the pattern of pharmacokinetic decay that the practitioner wishes to achieve. For example, dosing from one-four times a week is contemplated. The dosing regimen can vary over time. As used herein, a “subject” refers to any mammal, including humans and nonhumans, such as primates. Typically the subject is a human. A subject in need of identifying the presence of APOE4-related disorder phenotype is any subject at risk of, or suspected of, having APOE4-related disorder. A subject at risk of having an APOE4-related disorder may be a subject having one or more risk factors for APOE4-related disorder. Risk factors for APOE4-related disorder include, but are not limited to, age, family history, heredity and brain injury. In one embodiment, a subject at risk of having an APOE4-related disorder has one or more APOE4 alleles. In another embodiment, a subject at risk of having an APOE4-related disorder has two APOE4 alleles. Other risk factors will be apparent the skilled artisan. A subject suspected of having APOE4-related disorder may be a subject having one or more clinical symptoms of APOE4-related disorder. A variety of clinical symptoms of APOE4-related disorder are known in the art. Examples of such symptoms include, but are not limited to, memory loss, depression, anxiety, language disorders (eg, anomia) and impairment in their visuospatial skills.

In some embodiments, the subject has an APOE4-related disorder. In some embodiments, the subject has an APOE4-related disorder and is undergoing a putative treatment for an APOE4-related disorder. The methods described herein may be used to supplement the efficacy of a putative therapy for an APOE4-related disorder, i.e., for increasing the responsiveness of the subject to a putative therapy for an APOE4-related disorder. Based on this evaluation, the physician may continue the therapy, if there is a favorable response, or discontinue and change to another therapy if the response is unfavorable.

As used herein, the term “treating” refers to the application or administration of a composition including one or more active agents to a subject, who has a neurodegenerative disease, a symptom of a neurodegenerative disease, or a predisposition toward a neurodegenerative disease, with the purpose to cure, heal, alleviate, relieve, alter, remedy, ameliorate, improve, or affect the disorder, the symptom of the disease, or the predisposition toward a neurodegenerative disease.

Alleviating a neurodegenerative disease includes delaying the development or progression of the disease, or reducing disease severity. Alleviating the disease does not necessarily require curative results. As used therein, “delaying” the development of a disease (such as AD) means to defer, hinder, slow, retard, stabilize, and/or postpone progression of the disease or the development of plaques. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a neurodegenerative disease includes initial onset and/or recurrence.

In some embodiments, the choline supplementation is administered to a subject in need of the treatment at an amount sufficient to enhance synaptic memory function by at least 20% (e.g., 30%, 40%, 50%, 60%, 70%, 80%, 90% or greater). Synaptic function refers to the ability of the synapse of a cell (e.g., a neuron) to pass an electrical or chemical signal to another cell (e.g., a neuron). Synaptic function can be determined by a conventional assay.

Conventional methods, known to those of ordinary skill in the art of medicine, can be used to administer the pharmaceutical composition to the subject, depending upon the type of disease to be treated or the site of the disease. Preferably the choline supplementation is administered orally. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Treatment efficacy can be assessed by methods well-known in the art, e.g., monitoring synaptic function or memory loss in a patient subjected to the treatment.

It may be contemplated that the methods of the present invention may be used in combination with other drugs in the treatment of APOE4-related disorders. Examples of combinations of the methods of the present invention with other drugs in either unit dose or kit form include combinations with: anti-Alzheimer's agents, beta-secretase inhibitors, gamma-secretase inhibitors, HMG-CoA reductase inhibitors, NSAID's including ibuprofen, N-methyl-D-aspartate (NMDA) receptor antagonists, such as memantine, cholinesterase inhibitors such as galantamine, rivastigmine, donepezil, and tacrine, vitamin E, CB-1 receptor antagonists or CB-1 receptor inverse agonists, antibiotics such as doxycycline and rifampin, anti-amyloid antibodies, or other drugs that affect receptors or enzymes that either increase the efficacy, safety, convenience, or reduce unwanted side effects or toxicity of the compounds of the present invention. The foregoing list of combinations is illustrative only and not intended to be limiting in any way.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

A novel connection between the Alzheimer's Disease risk allele APOE4 and an increased requirement for choline to maintain lipid homeostasis was identified. A combination of lipidomics, unbiased genome-wide screens, as well as functional and genetic characterization was used to uncover that APOE4 induces widespread changes in lipid homeostasis in human induced pluripotent stem cell (iPSC) derived glia. Genetic and chemical modulators of these lipid disruptions were identified. In particular, it was discovered that supplementation with choline, a soluble phospholipid precursor is sufficient to dramatically rebalance the APOE4 lipidome, allowing these cells to behave more like APOE3 controls. Model organism genetics was used to characterize exactly how cells are utilizing the supplemented choline to achieve this rescue, and have demonstrated that in mouse models bearing human APOE4 that the results translate to effective reduction of Alzheimer's Disease relevant pathologies. This discovery provides a rationale for how environmental intervention such as increasing choline intake may improve glial health and stress buffering capacity, amyloid clearance, and may reduce inflammation. Ultimately, application of choline supplementation to APOE4 carriers may slow the rate of progression of AD and other diseases for which APOE4 is a risk factor.

### Example 1: Lipid Composition of APOE4 Astrocytes Relative to APOE3 Astrocytes

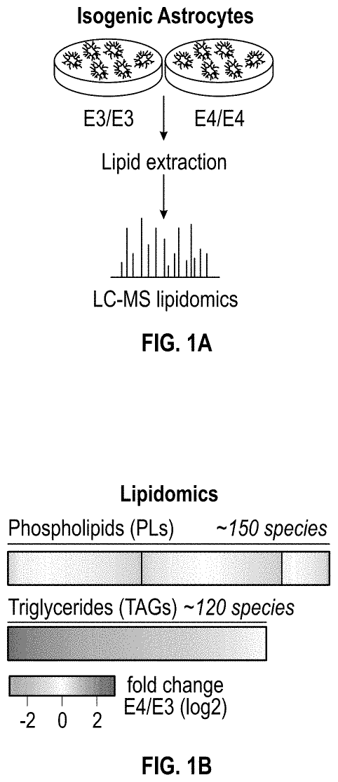

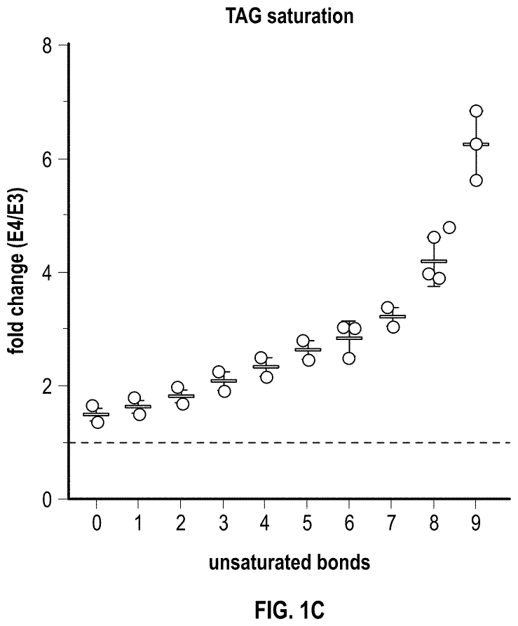

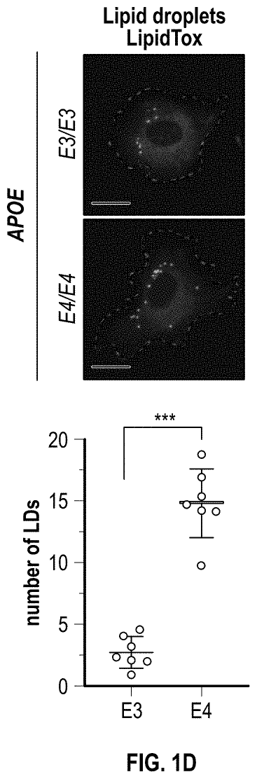

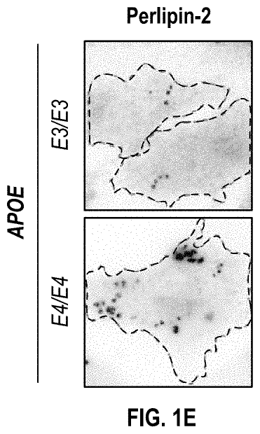

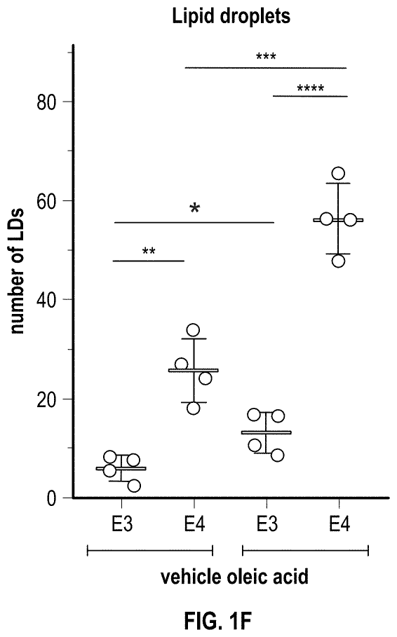

APOE is expressed in several organs, with the highest expression in the liver, followed by the brain. In the brain, astrocytes and to some extent microglia are the major cell types that express APOE in the brain (Kim, Basak et al. 2009). It was hypothesized that APOE4-mediated lipid dysregulation contributes to its role as a disease risk factor. Therefore, the lipidome of APOE4-expressing cells, focusing on the human brain cell type that produces the most APOE, astrocytes was characterized (Zhang et al, 2016). Using isogenic iPSCs differing only at the APOE locus, APOE3 or APOE4 astrocytes was generated (Lin et al, 2018). The lipid composition of the APOE3 and APOE4 astrocytes was compared using liquid chromatography-mass spectrometry (LC-MS) (FIG. 1A) (Ejsing et al, 2009). APOE4 astrocytes showed a profound increase in TAGs (FIG. 1B), and these had an increased number of unsaturated bonds (FIG. 1C) than the isogenic APOE3 astrocytes. TAGs, along with other neutral lipids such as cholesterol esters, are stored in specialized cytoplasmic organelles called lipid droplets (LDs). It was questioned whether the excess TAGs in the APOE4 astrocytes were contained in LDs using a lipophilic dye, LipidTox that stains neutral lipids. APOE4 astrocytes accumulate ˜3-fold more lipid droplets than their APOE3 counterparts (FIG. 1D). In addition, it was observed that a concomitant accumulation of a lipid droplet-resident protein, Perilipin-2 (FIG. 1E).

Lipid droplets not only act as a reservoir of energy or membrane biosynthesis but also protect from lipotoxicity by sequestering free fatty acids. Therefore, it was tested whether higher unsaturated fatty acid burden rendered APOE4 cells more sensitive to excess unsaturated fatty acids, such as oleic acid. Addition of oleic acid to APOE3 astrocytes increased their lipid droplet content by ˜1.5 fold. However, APOE4 astrocytes exposed to the same level of oleic acid exhibited an exacerbated lipid droplet accumulation (˜3 fold) (FIG. 1F). Together these data demonstrate that APOE4 astrocytes accumulate excess TAGs, stored in LDs, and they have reduced ability to buffer exogenous lipid stress.

### Example 2. Molecular Mechanism of APOE4-Mediated Lipid Dysfunction

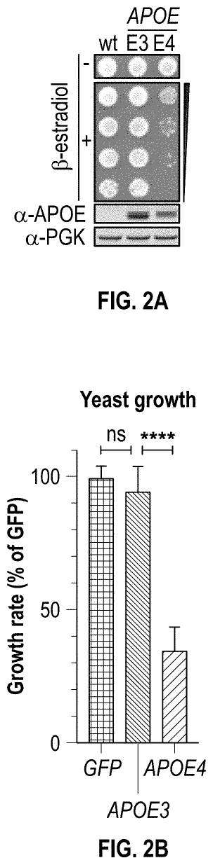

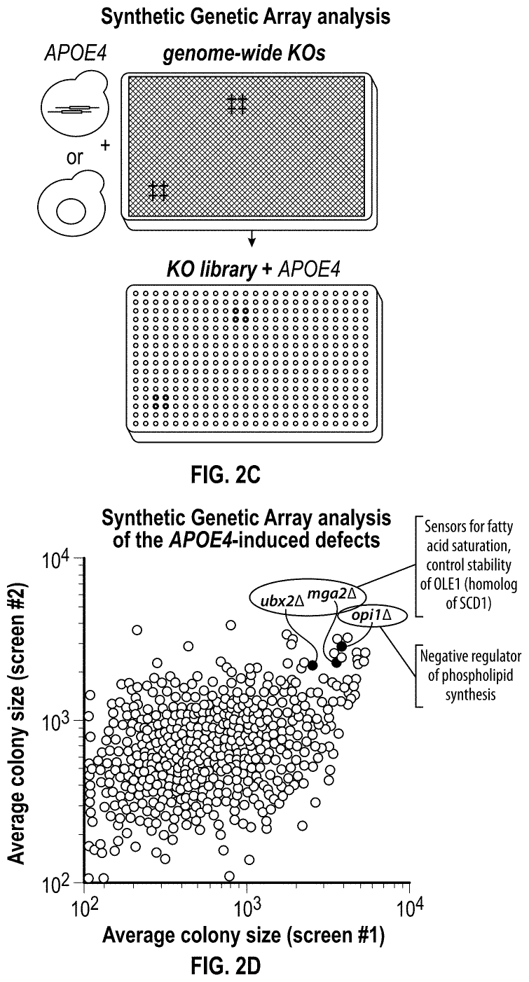

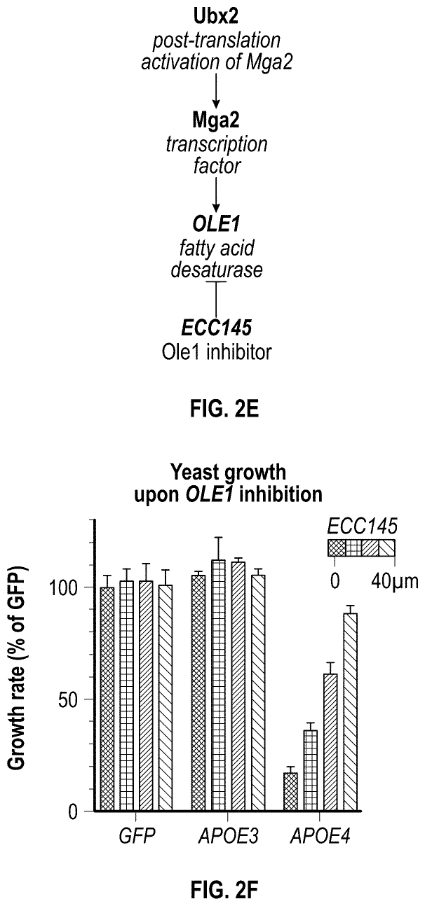

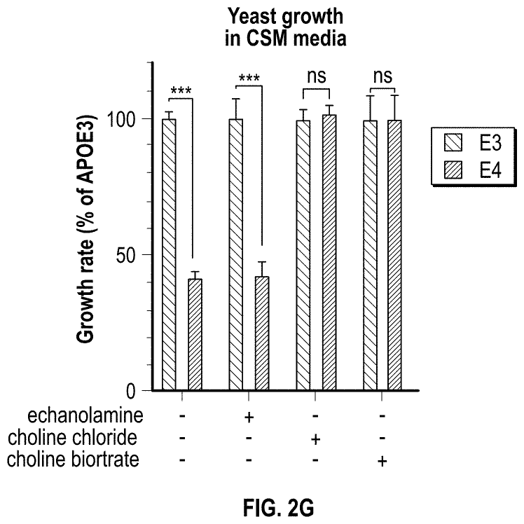

In order to explore APOE4-mediated lipid dysregulation in an unbiased manner, yeast were built and interrogated that express APOE3 or APOE4 in to the secretory pathway. It was confirmed that yeast APOE4 show similar defects in lipid homeostasis, including accumulation lipid droplets and TAG (data not shown), as well as a growth defect (FIG. 2A, quantified in FIG. 2B). A genetic screening in this model was performed (FIG. 2C), and determined that deletions in key sensors for fatty acid saturation, and an inhibitor of phospholipid synthesis (FIG. 2D) (Klig et al, 1985; Surmaet et al, 2013; Schuldiner et al, 2005) could rescue the APOE4 defects. These data suggested key genetic nodes that modify APOE4 toxicity: modulation of fatty acid saturation status, and phospholipid synthesis. These pathways were independently confirmed as relevant to APOE4 toxicity through chemical targeting or media supplementation. First, chemical targeting of lipid desaturase OLE1, the yeast homolog of human Stearyl co-A Desaturase (SCD) showed dose-dependent rescue of APOE4 growth rate (FIG. 2E-F), validating the identification of Ubx2 and Mga2 as modulators of APOE4 toxicity. Since one of the top genetic screen hits, OPI1 is a negative regulator of phospholipid synthesis, soluble precursors of phospholipid synthesis, ethanolamine and choline, were supplemented into the CSM to stimulate phospholipid synthesis. While ethanolamine did not influence growth of the cells expressing the APOE4 gene, addition of choline salts (choline chloride or choline bitartrate) to minimal yeast media (CSM) was sufficient to suppress the APOE4-associated growth defect (FIG. 2G). Supplementation of yeast media with choline also normalized yeast TAG levels and extent of saturation, without suppressing APOE4 expression (data not shown). These results suggest that the benefits of choline and CDP-choline are related to the Kennedy Pathway synthesis of phosphatidylcholine.

### Example 3. Choline Rescues APOE4 Defects in Human Cell Culture

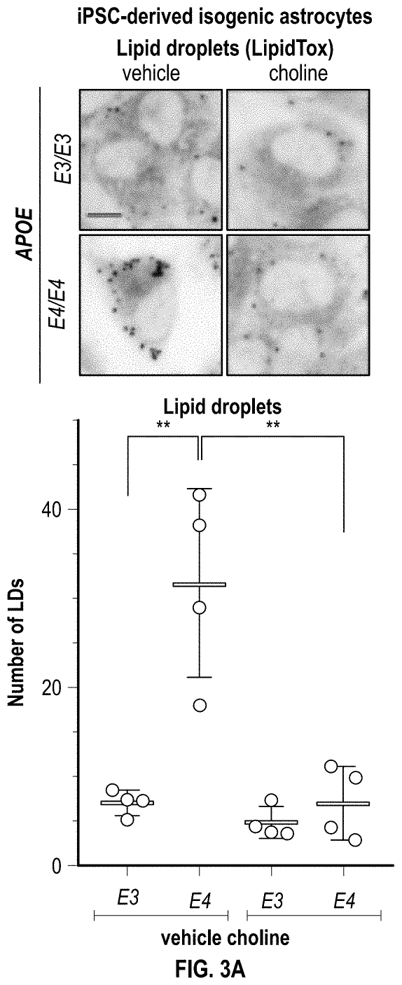

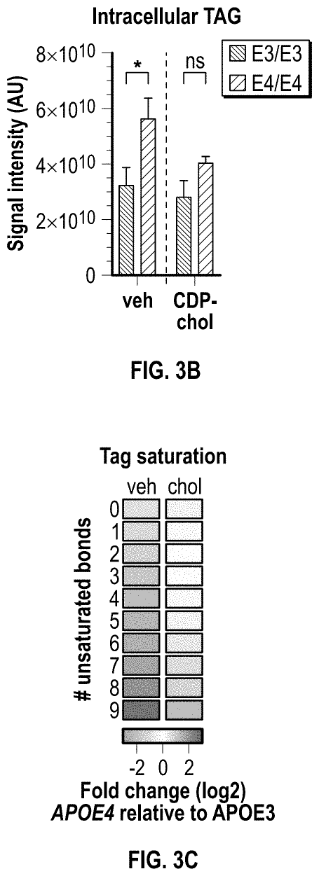

The conservation of these effects in human cells was observed. Chemical inhibitors, including inhibitors targeting lipid saturation or accumulation of TAG from precursors, reduces the accumulation of lipid droplets in APOE4 astrocytes, confirming that similar pathways are engaged in human astrocytes as we discovered in yeast (data not shown). Importantly, it was also found that APOE4 astrocytes grown in media supplemented with choline chloride or CDP-choline, which is a direct precursor in the synthesis of PC by the Kennedy pathway, showed a significant decrease in the LD number, down to the levels found in APOE3-expressing astrocytes (FIG. 3A). Furthermore, it was found that while vehicle-treated APOE4 astrocytes display an increased level of TAGs compared to APOE3, CDP-choline treatment normalizes TAG levels (FIG. 3B) and their unsaturation (FIG. 3C). These results indicate that choline supplementation ameliorates APOE4-induced lipid defects in iPSC-derived human astrocytes.

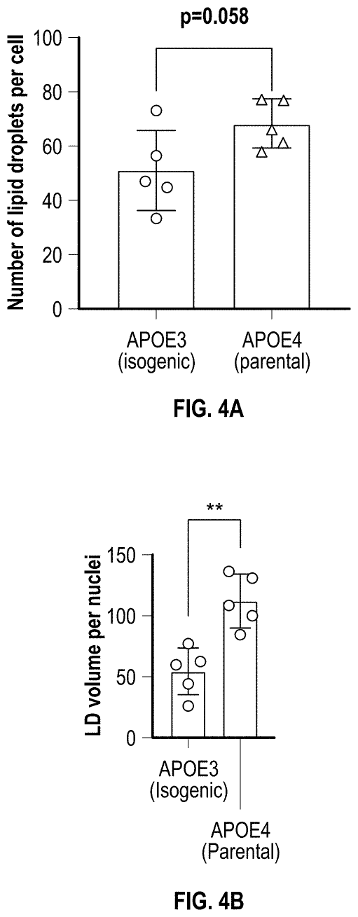

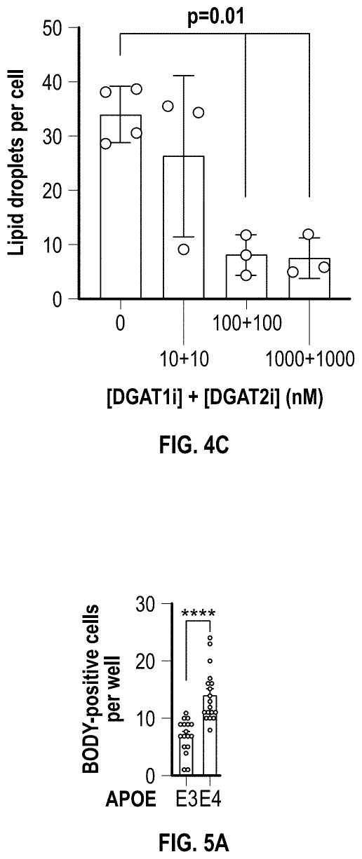

Critically, key phenotypes and recues were independently validated in a second isogenic pair of APOE3 and APOE4 astrocytes derived from another donor, including lipid accumulation in lipid droplets (FIG. 4A-B) and rescue with chemical inhibitors (FIG. 4C). These data demonstrate that the defects that have been identified and characterized associated with APOE4 are due to the presence of the APOE4 allele, and independent of genetic background.

### Example 4: Lipid Dysfunction in APOE4 Microglia

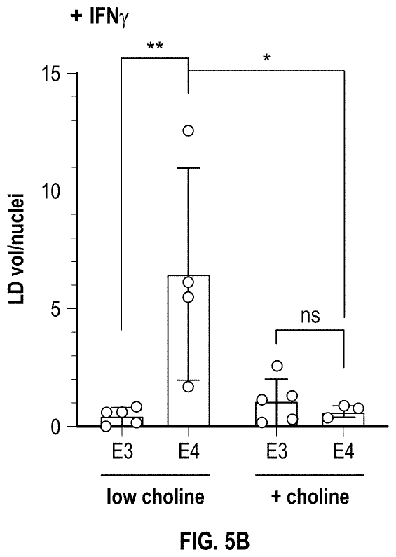

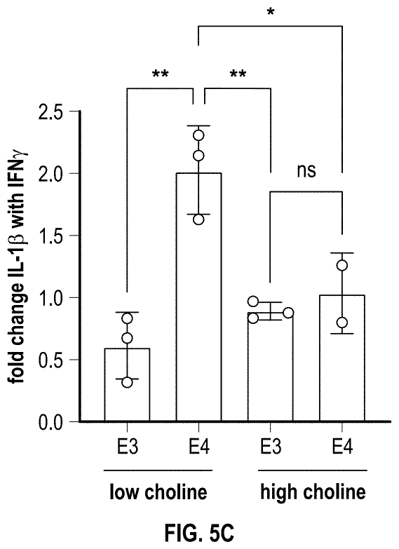

Many AD risk factors are expressed in microglia including APOE, which along with TREM2, coordinates the transition from homeostatic to disease-associated state (Kraseman et al, 2017; Keren-Shaul et al, 2017). Indeed iPSC-derived APOE4 microglia display impaired phagocytosis, migration and metabolic activity, as well as exacerbated cytokine secretion (9,38). It was examined whether APOE4 microglia also display disrupted lipid homeostasis, and found that indeed APOE4 iPSC-derived microglia accumulate more lipid droplets under standard culturing conditions (FIG. 5A). It was also observed that APOE4 displayed increased lipid droplet volume following extended culture in choline limiting media and activation with interferon gamma, compared to isogenic APOE3 microglia (FIG. 5B, low choline). Importantly, this defect can be attenuated by supplementing the media with choline (FIG. 5B, +choline). These microglia also show higher levels of Il-1b induction following activation with interferon gamma compared to APOE3, and that this induction can also be attenuated with choline (FIG. 5C). These data suggest the possibility that choline supplementation may normalize microglial activation in APOE4 carriers.

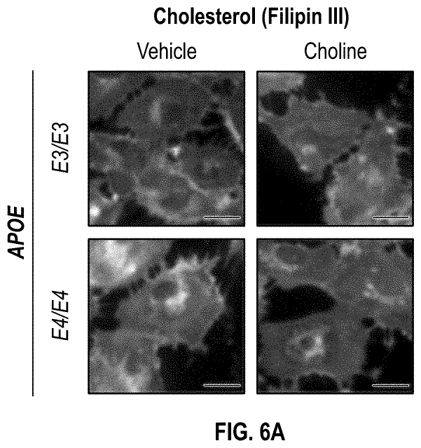

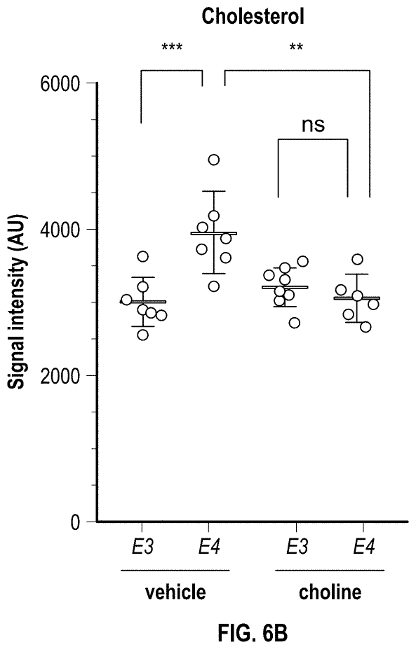

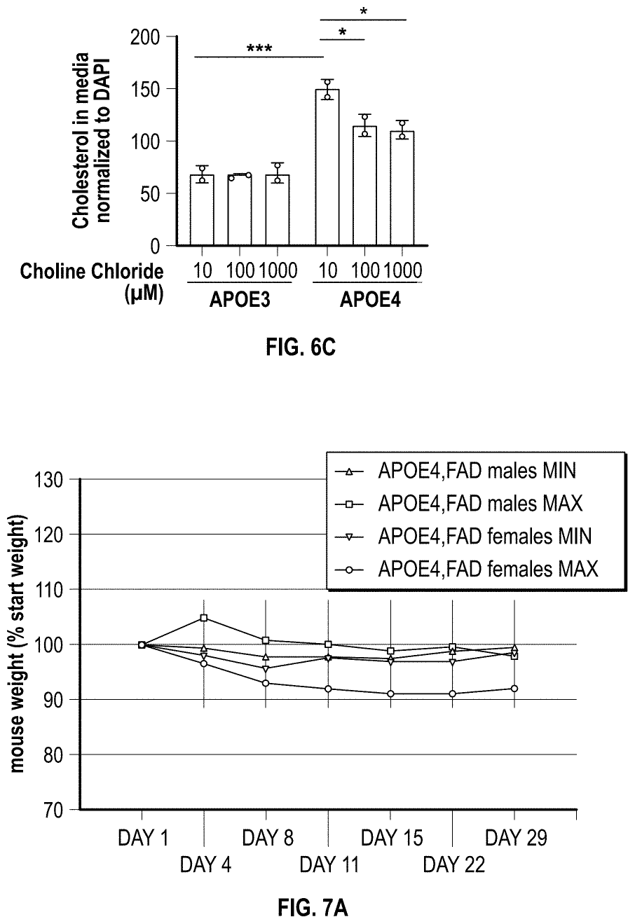

APOE4 also increases cholesterol content in astrocytes as measured by Filipin III under standard culturing (Lin et al, 2018) and extended culturing conditions. Following culture in media containing supplemented choline, the cholesterol intensity in APOE4 is no longer significantly different from control APOE3 (FIG. 6A-B). Increased cholesterol in the media of APOE4 astrocytes that were previously reported were compared to controls (Lin et al, 2018). This defect is also ameliorated by choline in a dose dependent manner (FIG. 6C).

### Example 5. Choline Supplementation in Animal Models of AD

Previous evidence suggests that AD mouse models can respond to variation in choline dietary levels. Maternal, perinatal, and lifelong dietary choline supplementation all improve various endpoints such as neuronal plasticity, behavioral deficits, microglial activation, and/or amyloid pathology in multiple models of Down Syndrome and AD (Kelley et al, 2019; Velasquez et al, 2019; Mellott et al, 2017; Wang et al, 2019). However, dietary choline has not been studied applied exclusively in adulthood, and never in a humanized APOE genetic background. It is now sought to understand how dietary choline might modify APOE mouse models, both with and without transgenic backgrounds that ensure accumulation of AD-relevant pathologies such as amyloid.

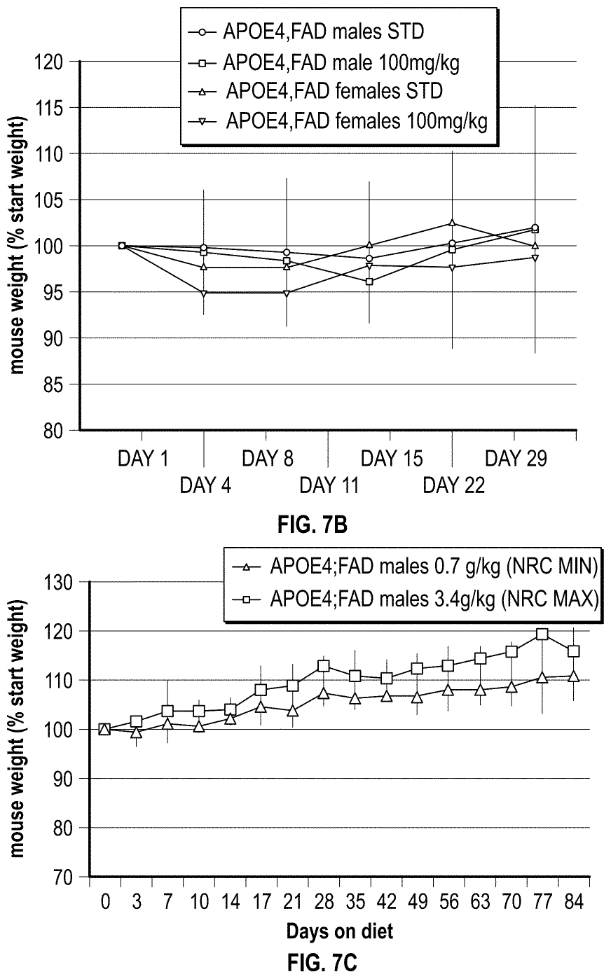

The “EFAD” APOE knock-in mouse were selected, where the endogenous Apoe locus is replaced with the human isoform of APOE2, APOE3, or APOE4 in 5×FAD mice, and where APOE isoform effects on disease progression have been documented (Tai et al, 2017). Custom chow containing the National Research Council (NRC) minimum, (0.7 g/kg choline chloride) and NRC maximum (3.4 g/kg choline chloride) was manufactured and fed these to EFAD mice for 4-12 weeks (˜84 days). There was no clear indication of toxicity and general health appeared normal on both diets (FIGS. 7A-7C).

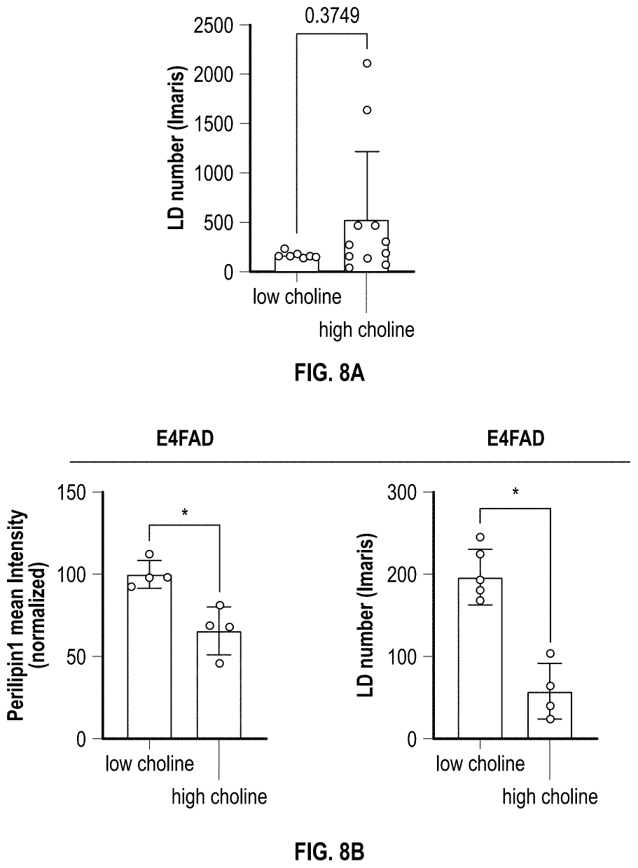

It was assessed whether E4FAD animals exhibited lipid defects compared to E3FAD animals. Using an antibody against lipid droplet associated protein, perilipin-1, a trend to increased perilipin-1 in the dentate gyrus (DG) of E4FAD animals compared to E3FAD was detected (FIG. 8A), consistent with reported results in human iPS-derived astrocytes. It was then evaluated whether 3 months of high choline diet could modify these effects. Indeed, high choline diet is associated with a significant reduction in perilipin-1 mean intensity and presumed lipid droplet count (FIG. 8B) in animals fed high choline (3.4 g/kg) for 3 months compared to those fed a low choline diet (0.7 g/kg) for the same length of time.

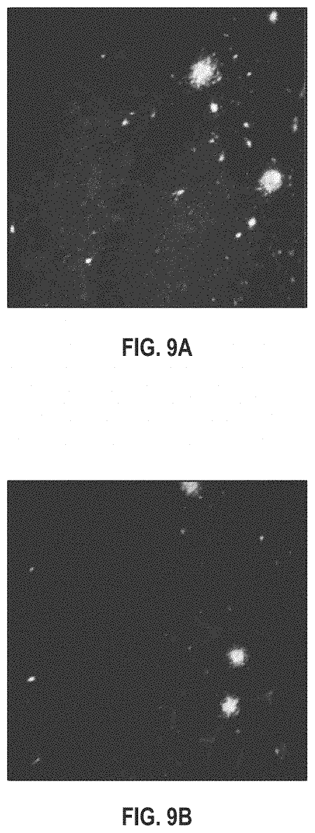

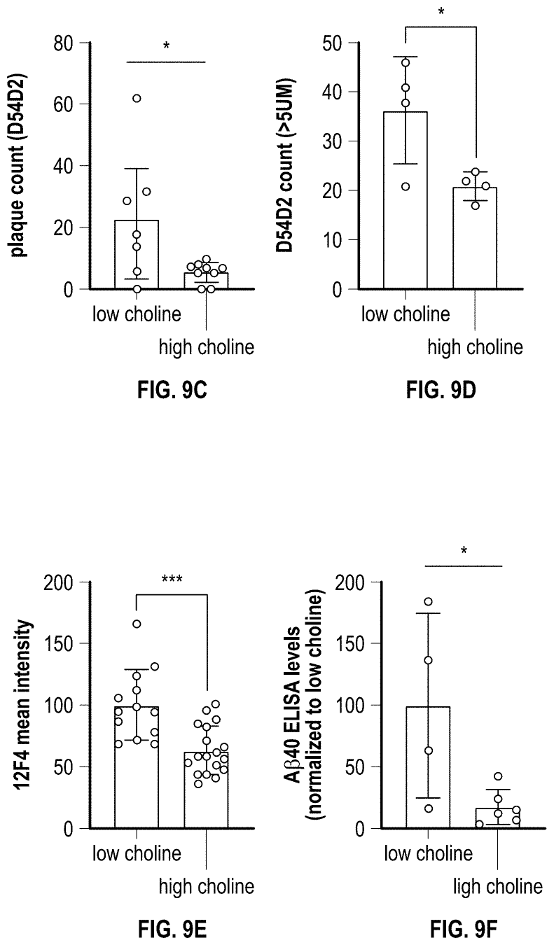

It is currently being assessed how these diets impact AD-relevant outcomes in the EFAD model. Given previous reports of APOE4 impacting amyloid pathology, the hippocampus and cortex of animals were examined on low and high choline diet by ELISA. Encouragingly, high choline is associated with decreased amyloid load in multiple regions of the hippocampus. There was reduced amyloid in the dentate gyrus (DG) of female animals fed high choline diet (FIG. 9B) compared to those fed low choline diet (FIG. 9A). These results are quantified in FIG. 9C. Male mice, which generally have less aggressive pathology in this model, similarly showed reduction of amyloid burden in the CA1 region of the hippocampus (FIG. 9D). Staining for multiple amyloid markers in fixed brains of females following 3 months of diet in cortex and hippocampus shows that there is also a significant reduction in amyloid load in the CA1 region of animals on high choline using the independent 12F4 antibody (FIG. 9E). Enzyme-linked immunosorbent assay (ELISA) for Aβ40 levels also showed significant reduction in the cortices of female mice fed high choline diet compared to low choline diet (FIG. 9F). These results suggest that choline supplementation might alter brain amyloid metabolism, which is a highly APOE-dependent process.

Finally, an unbiased approach to determine the effect of high choline diet on E4FAD mice was employed to determine the biological pathways relevant to disease that are modified by nutrient supplementation. The powerful technique of Fluorescent Activated Nuclear Sorting (FANS) was harnessed, whereby the nuclei of specific cell types, such as neurons, astrocytes, microglia and oligodendrocytes were isolated from mouse brain tissue (Marion-Poll et al, 2014). Following isolation by FANS, RNA-sequencing analyses in these neural cell subtypes to observe genes that are up- or down-regulated in E4FAD mouse brains in response to 3 months of high- or low-choline diet were performed. These data will reveal how choline is modifying our APOE4 carrying AD mouse model in complex tissue, with cell type specific detail, and suggest to us how choline supplementation may impact a human brain.

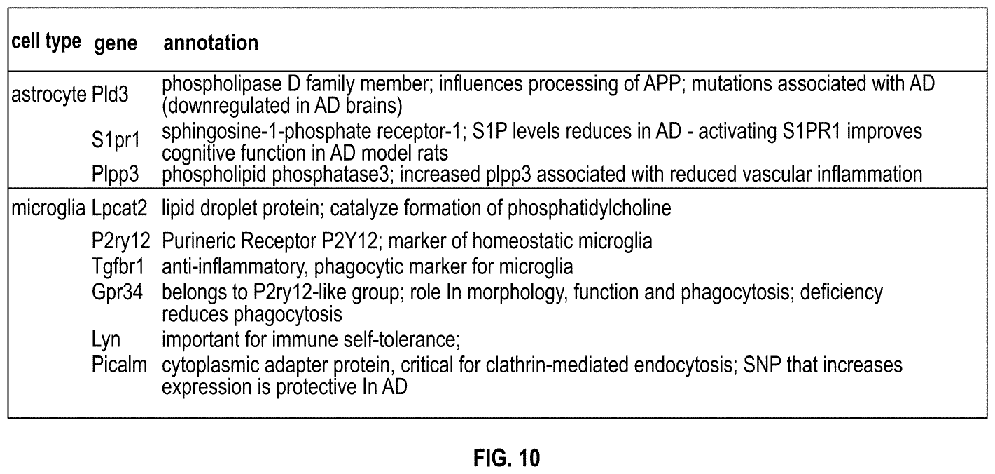

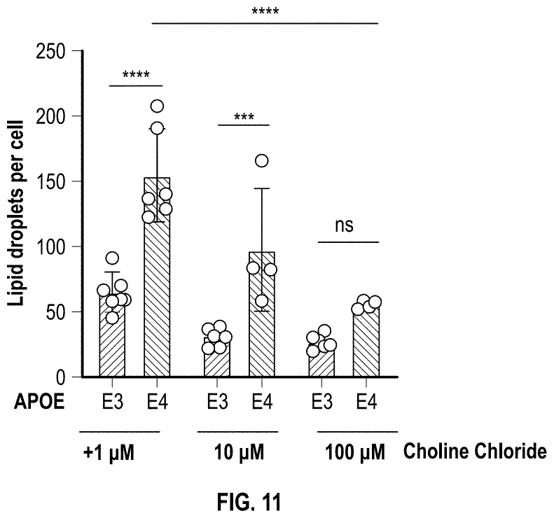

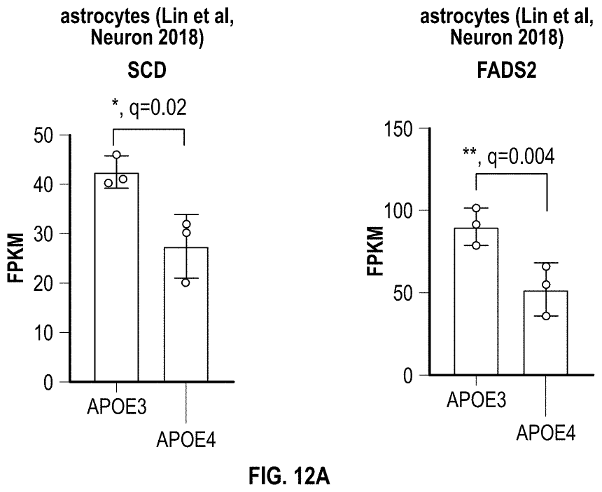

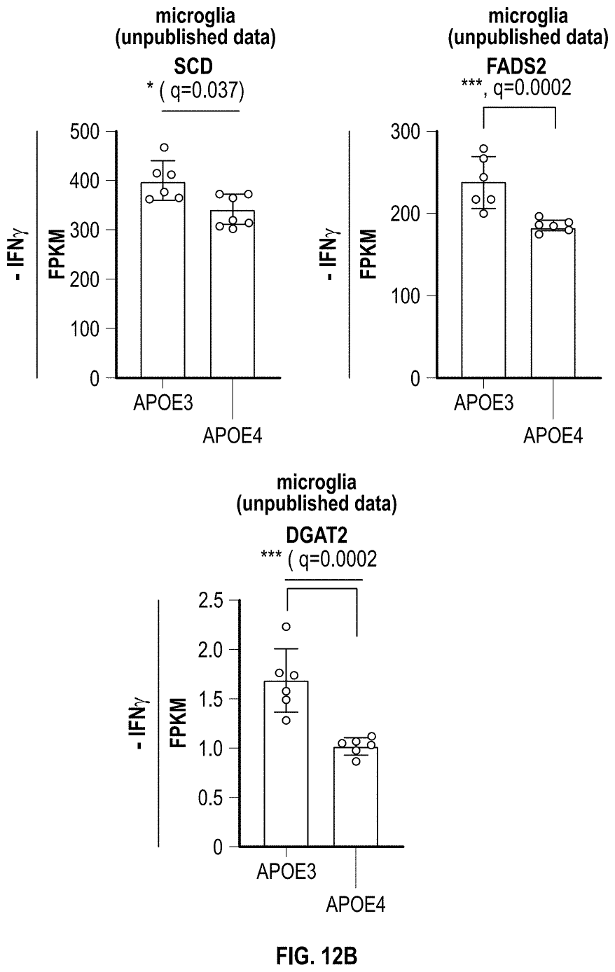

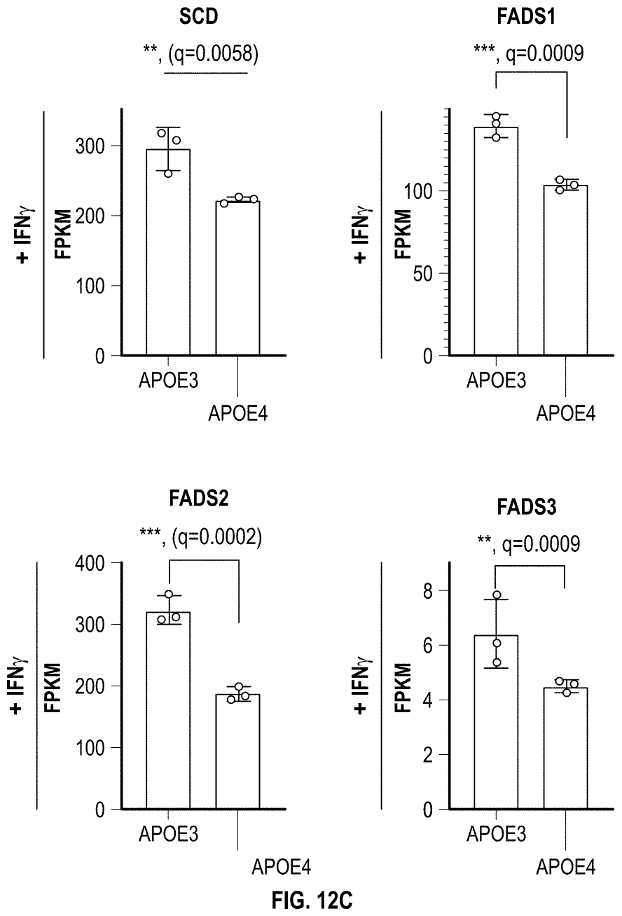

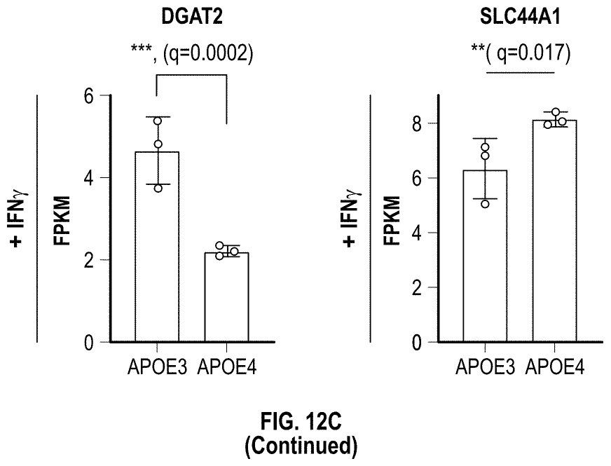

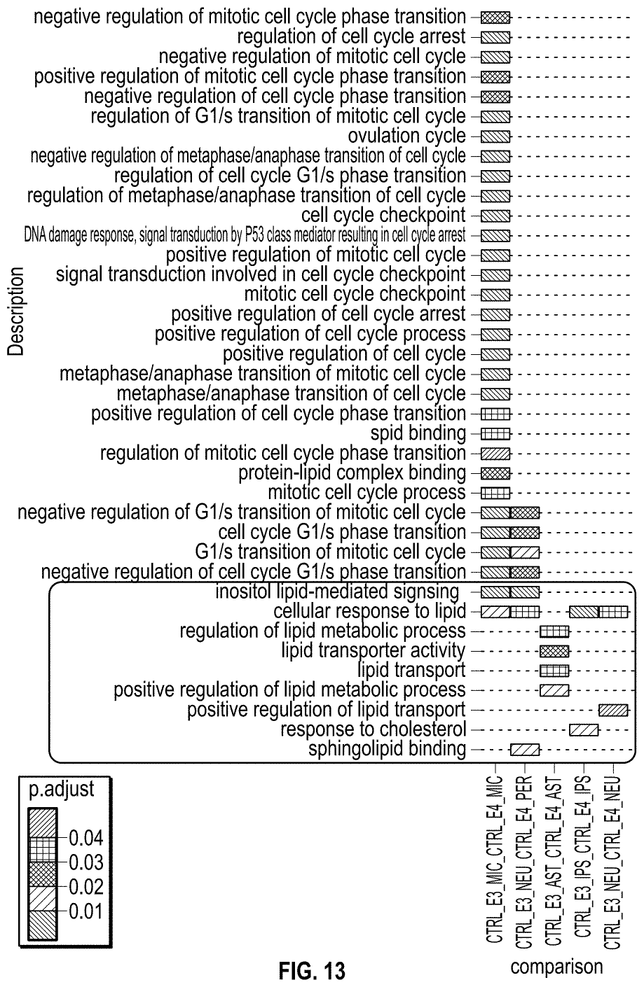

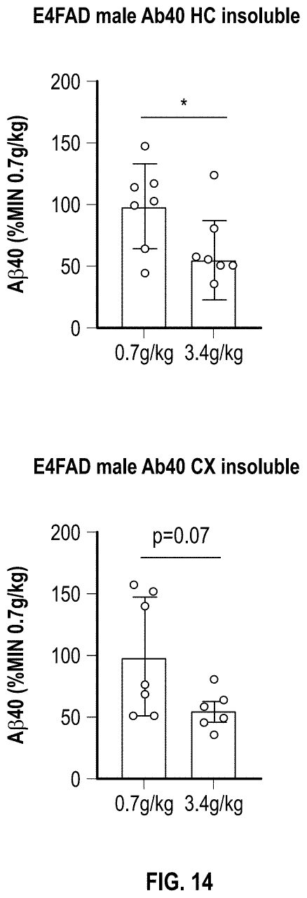

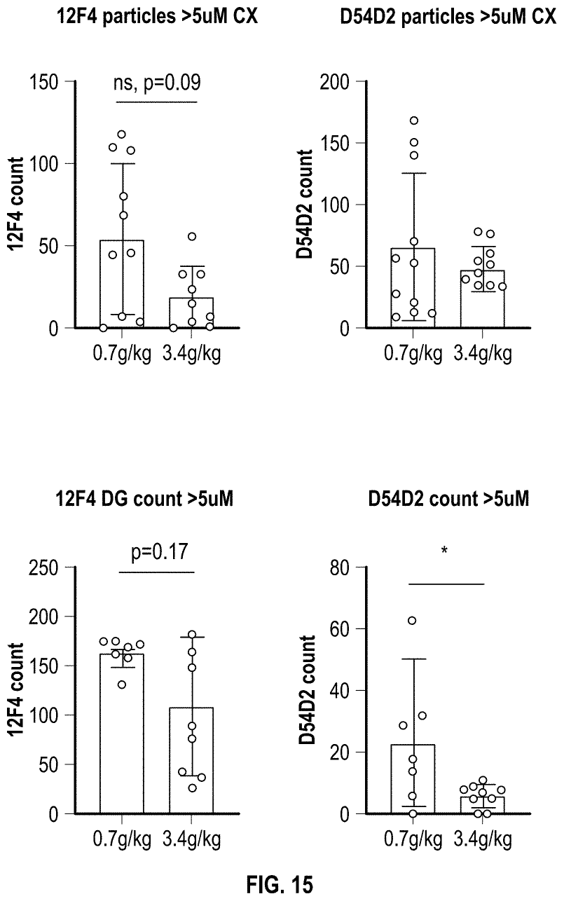

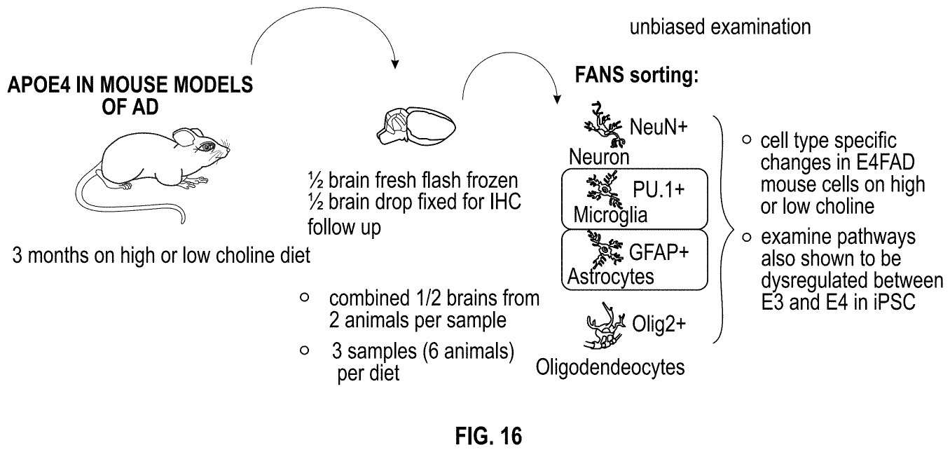

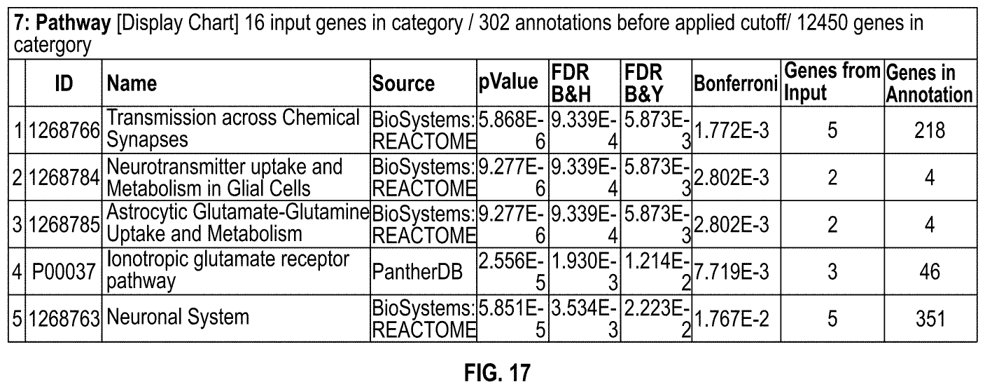

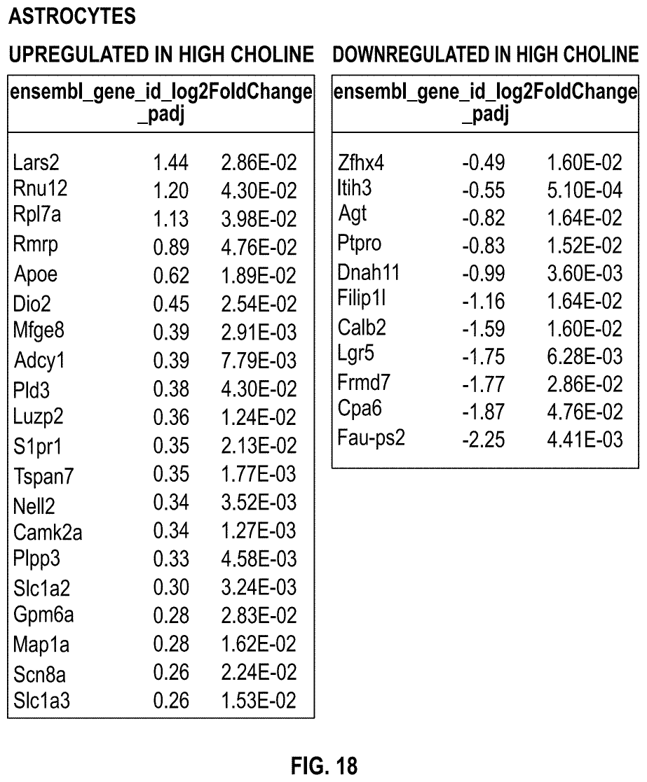

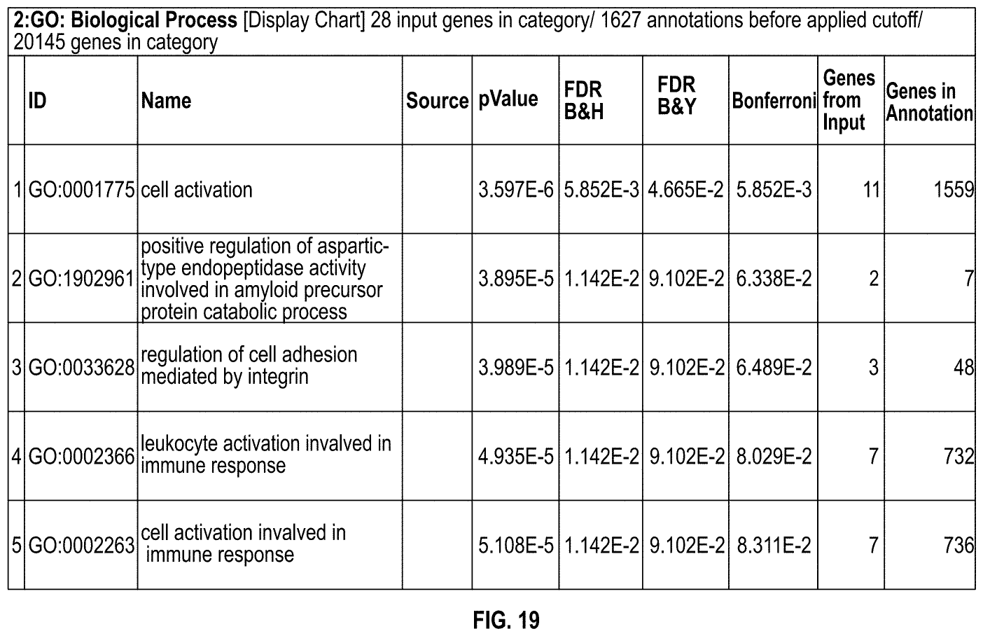

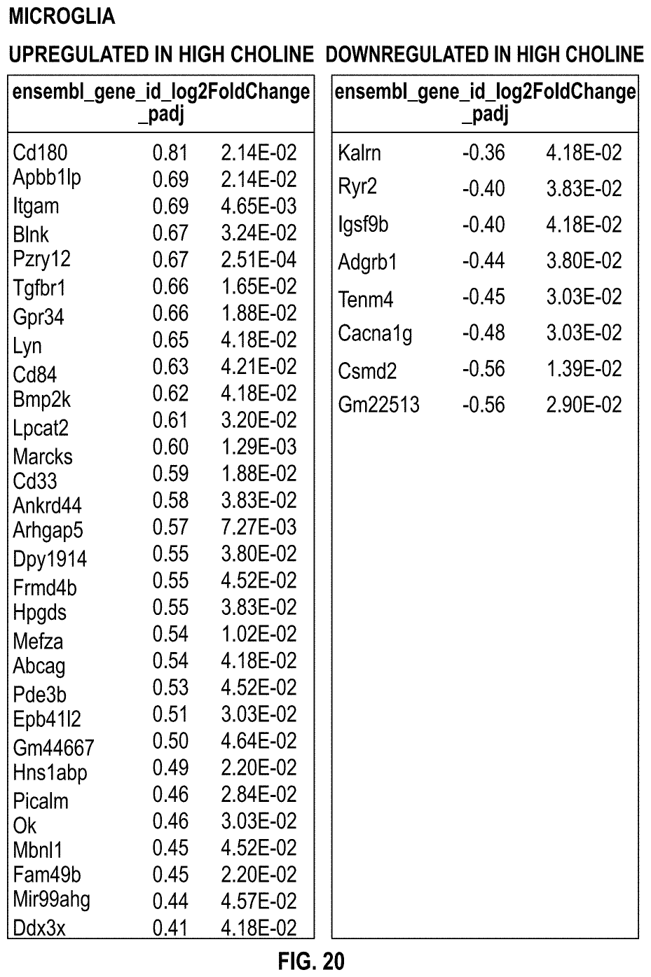

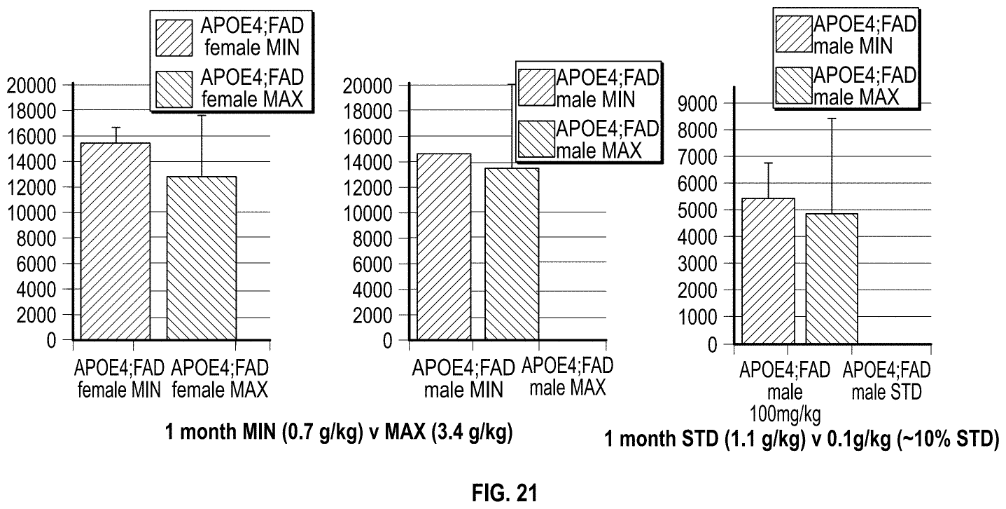

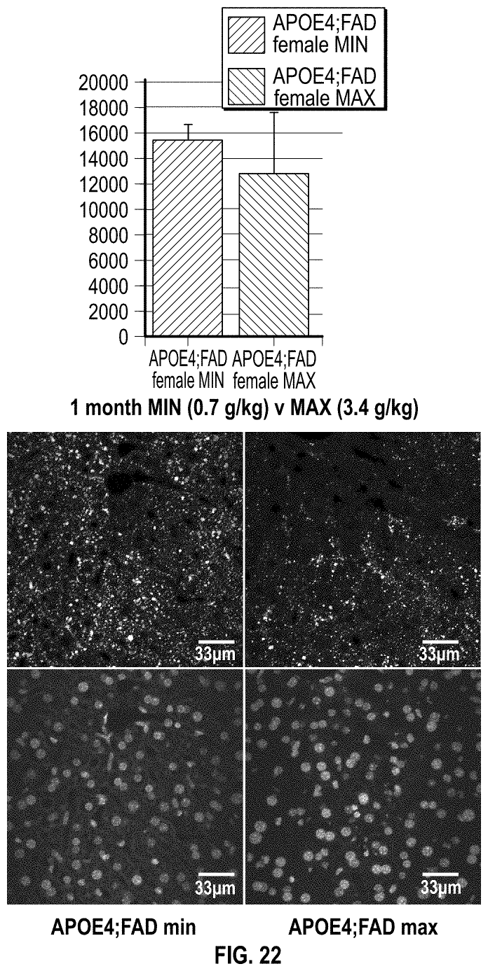

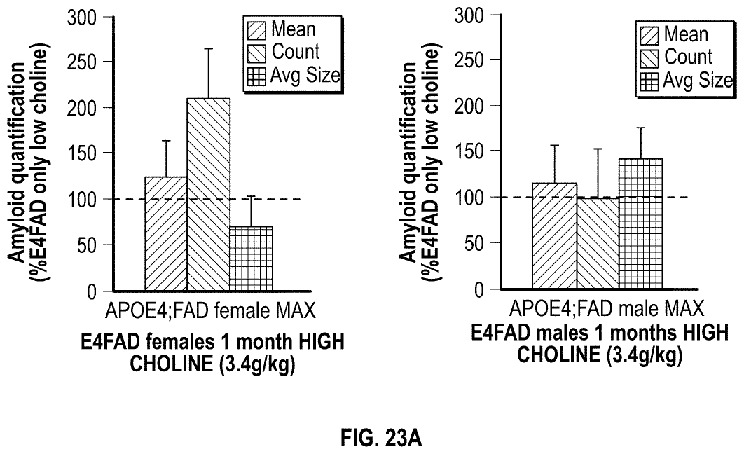

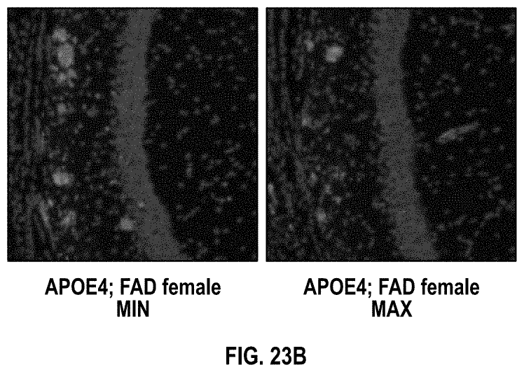

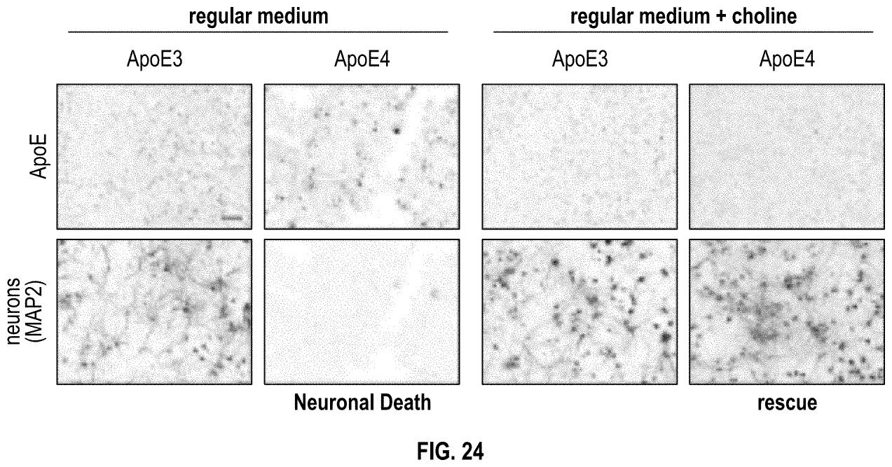

The cortical tissue in females was examined first, as this region displayed a significant reduction in Aβ levels by ELISA (FIG. 9F). Both astrocytes and microglia were isolated from the cortex of female E4FAD fed low- or high-choline diet for 3 months (FIG. 16). In both astrocytes and microglia isolated from female cortices of mice fed high or low choline diet, significant changes were found in multiple genes relevant to the pathways that have been identified in the astrocyte experiments described above (Examples 1-3, FIG. 10). For instance, changes in PC metabolic genes, such as an increase in Lpcat2 in microglia, which is a lipid droplet-associated enzyme that supports PC synthesis were identified. Interestingly, changes in several genes that are both involved in PC metabolism and previously have been associated with AD risk or disease progression were identified. For instance, it was observed that Pld3, one of the rare AD GWAS hits, is upregulated in astrocytes of female E4FAD cortex on high choline diet compared to low choline diet. Phospholipase D hydrolyzes PC into phosphatidic acid, and mutations in this gene that downregulate expression are associated with AD (Lambert et al, 2015). Similarly, in astrocytes induction of Plpp3, which hydrolyzes phosphatidic acid into diacylglycerol and is associated with lower vascular inflammation (Busnelli et al, 2018), and the sphingosine-1-phosphate (S1P) receptor 1, which recognizes the neuroprotective PC derivative S1P to modulate cognitive function (Couttas et al, 2014) was also shown. Together, these data support the hypothesis that changes in PC metabolism will be detectable after choline supplementation.

Strikingly, many of the genes identified as upregulated in microglia from E4FAD females on high choline chow are markers of non-inflammatory homeostatic microglia, such as P2ry12, Tgfbr1, and Gpr34, suggesting that dietary choline may be attenuating inflammation in E4FAD animals. These data together support the earlier preliminary data in iPSCs that choline supplementation could affect the state of inflammation in the CNS.

Examination of other changes observed in astrocytes in high choline compared to low choline revealed that a number of glutamate receptors and transporters were upregulated in E4FAD astrocytes of animals fed high choline diet compared to low choline diet (Slc1a2, Slc1a3). These high affinity glutamate transporters represent the most important mechanism for removal of glutamate from the extracellular space, preventing glutamate excitotoxicity and acting as a vital component of plasticity and synaptic function (Rose et al, Front Mol Neurosci, 2018). The upregulation of these transporters in astrocytes of mice fed high choline, therefore, may protect against neuronal damage and improve neuronal outcomes.

Non-traditional AD pathology outcomes, such as changes in myelination were also explored. Increased white matter damage has been observed for APOE4 mice (Koizumi et al, Nat Commun, 2018). Preliminary data suggests that APOE4 animals fed high choline diet show increased myelination compared to animals fed low choline diet (data not shown). These preliminary results suggest that increased dietary choline may improve multiple neuronal health outcomes.

In summary, a novel molecular pathway specifically affected by APOE4 status has been identified, it has been discovered that choline supplementation normalizes the APOE4-mediated dysregulation, and it has been validated this concept in human model systems and in vivo in an AD mouse model. The data presented here suggest amyloid deposition and turnover, PC metabolism, and synaptic health and inflammation may be modified in APOE4 carriers given choline supplementation for the appropriate dose and timeframe.

### Example 6. Advantages and Improvements Over Existing Methods, Devices or Materials

Choline-esterase and supplemental choline have been applied in various contexts including AD (Gareri, Castagna et al. 2015), yet the connection between choline deficiency and the APOE4 specific genotype that has been identified is completely novel. While the APOE4 genotype is enriched in AD populations relative to the general population, treatment paradigms have largely not been stratified for APOE allele status, and are thus not likely representative of the beneficial effects of choline supplementation specific to APOE4 carriers. Moreover, specific treatment paradigms may be required to re-establish lipid homeostasis in APOE4 carriers independent of other benefits of generic choline application. These conditions will be determined in mammalian experiments currently underway in both human iPS and mouse models of AD.

The careful characterization of these APOE4-specific phenotypes provides several valuable readouts by which to assess the success of choline supplementation. It is anticipated that several other readouts based on the mouse model experiments will be identified. These outcomes will provide a much more sensitive and biochemically accessible understanding of the potential success of choline supplementation in human APOE4 carriers.

The approach is unique in that it unites two previously unconnected aspects of AD pathology, cognitive decline, and treatment: choline supplementation (perhaps in combination with choline esterase inhibition) and lipid dysregulation in APOE4 carriers. The novel finding that APOE4 creates an increased demand for choline, likely via an increased demand for phosphatidylcholine, has significant relevance to the treatment of APOE4-specific disease pathologies. Indeed, while studies have focused on AD relevant phenotypes, it is reasonable to hypothesize that the lipid dysregulation observed in yeast and human iPS-derived astrocytes would be true for any cell/tissue expressing or requiring APOE function. Indeed, as mentioned above, APOE4 is associated with multiple disorders across a range of tissues, including Cerebral Amyloid Angiopathy (CAA), cardiovascular diseases such as atherosclerosis, and recovery from traumatic brain injury (TBI). Dietary choline application, particularly preventative application, in these contexts would be hypothesized reduce pathologies induced by APOE4 across multiple tissue types.

APOE4 specific choline precursors and dosage recommendations that will alleviate the higher choline requirement in APOE4 carriers compared to the general public are contemplated. It is proposed that to patent the specific application of choline supplementation for APOE4 carriers, differentiating this application from the generic health benefit previously established for choline supplementation. The data suggests that choline supplement protects APOE4 carriers from disorders including CAA, cardiovascular disease and atherosclerosis, and sporadic Alzheimer's Disease, as well as protect neural integrity following traumatic brain injury (TBI). In the case of CAA, TBI, AD, and potentially other neurodegenerative disorders, it is contemplated that the cognitive capacity of APOE4 carriers will be protected by early intervention with specific choline therapies.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

### REFERENCES

Bu, G. (2009). “Apolipoprotein E and its receptors in Alzheimer's disease: pathways, pathogenesis and therapy.” Nat Rev Neurosci 10(5): 333-344.
Busnelli M, et al. Lipid phosphate phosphatase 3 in vascular pathophysiology. Atherosclerosis. 2018 April; 271:156-165.
Cohen, B. M., P. F. Renshaw, A. L. Stoll, R. J. Wurtman, D. Yurgelun-Todd and S. M. Babb (1995). “Decreased brain choline uptake in older adults. An in vivo proton magnetic resonance spectroscopy study.” JAMA 274(11): 902-907.
Couttas T A, et al. Loss of the neuroprotective factor Sphingosine 1-phosphate early in Alzheimer's disease pathogenesis. Acta Neuropathol Commun. 2014 Jan. 23; 2:9
Ejsing C. S., et al, Global analysis of the yeast lipidome by quantitative shotgun mass spectrometry, Proc. Natl. Acad. Sci. {U.S.A.} 106, 2136-2141 (2009).
Gareri, P., A. Castagna, A. M. Cotroneo, S. Putignano, G. De Sarro and A. C. Bruni (2015). “The role of citicoline in cognitive impairment: pharmacological characteristics, possible advantages, and doubts for an old drug with new perspectives.” Clin Intery Aging 10: 1421-1429.
Hamilton, L. K., M. Dufresne, S. E. Joppe, S. Petryszyn, A. Aumont, F. Calon, F. Barnabe-Heider, A. Furtos, M. Parent, P. Chaurand and K. J. Fernandes (2015). “Aberrant Lipid Metabolism in the Forebrain Niche Suppresses Adult Neural Stem Cell Proliferation in an Animal Model of Alzheimer's Disease.” Cell Stem Cell 17(4): 397-411.
Houlden, H. and R. Greenwood (2006). “Apolipoprotein E4 and traumatic brain injury.” J Neurol Neurosurg Psychiatry 77(10): 1106-1107.
Kelley C M et al. Maternal Choline Supplementation Alters Basal Forebrain Cholinergic Neuron Gene Expression in the Ts65Dn Mouse Model of Down Syndrome. Dev Neurobiol. 2019 July; 79(7):664-683
Keren-Shaul H, et al. A Unique Microglia Type Associated with Restricting Development of Alzheimer's Disease. Cell. 2017 Jun. 15;169(7):1276-1290
Kim, J., J. M. Basak and D. M. Holtzman (2009). “The role of apolipoprotein E in Alzheimer's disease.” Neuron 63(3): 287-303.
Klig L. S., M. J. Homann, G. M. Carman, S. A. Henry, Coordinate regulation of phospholipid biosynthesis in Saccharomyces cerevisiae: Pleiotropically constitutive opil mutant, J. Bacteriol. 162, 1135-1141 (1985).
Krasemann S, et al The TREM2-APOE Pathway Drives the Transcriptional Phenotype of Dysfunctional Microglia in Neurodegenerative Diseases. Immunity. 2017 Sep. 19;47(3):566-581.e9.
Lin, Y. T., J. Seo, F. Gao, H. M. Feldman, H. L. Wen, J. Penney, H. P. Cam, E. Gjoneska, W. K. Raja, J. Cheng, R. Rueda, O. Kritskiy, F. Abdurrob, Z. Peng, B. Milo, C. J. Yu, S. Elmsaouri, D. Dey, T. Ko, B. A. Yankner and L. H. Tsai (2018). “APOE4 Causes Widespread Molecular and Cellular Alterations Associated with Alzheimer's Disease Phenotypes in Human iPSC-Derived Brain Cell Types.” Neuron 98(6): 1141-1154 e1147.
Marion-Poll L, et al Fluorescence-activated sorting of fixed nuclei: a general method for studying nuclei from specific cell populations that preserves post-translational modifications. Eur J Neurosci. 2014 April; 39(7):1234-44.
Mellott T J, et al Perinatal Choline Supplementation Reduces Amyloidosis and Increases Choline Acetyltransferase Expression in the Hippocampus of the APPswePS 1dE9 Alzheimer's Disease Model Mice. PLoS One. 2017 Jan. 19;12(1)
Nitsch, R. M., J. K. Blusztajn, A. G. Pittas, B. E. Slack, J. H. Growdon and R. J. Wurtman (1992). “Evidence for a membrane defect in Alzheimer disease brain.” Proc Natl Acad Sci USA 89(5): 1671-1675.
Petersen, R. C., R. G. Thomas, M. Grundman, D. Bennett, R. Doody, S. Ferris, D. Galasko, S. Jin, J. Kaye, A. Levey, E. Pfeiffer, M. Sano, C. H. van Dyck, L. J. Thal and G. Alzheimer's Disease Cooperative Study (2005). “Vitamin E and donepezil for the treatment of mild cognitive impairment.” N Engl J Med 352(23): 2379-2388.
Rannikmae, K., N. Samarasekera, N. A. Martinez-Gonzalez, R. Al-Shahi Salman and C. L. Sudlow (2013). “Genetics of cerebral amyloid angiopathy: systematic review and meta-analysis.” J Neurol Neurosurg Psychiatry 84(8): 901-908.
Schmukler, E., D. M. Michaelson and R. Pinkas-Kramarski (2018). “The Interplay Between Apolipoprotein E4 and the Autophagic-Endocytic-Lysosomal Axis.” Mol Neurobiol 55(8): 6863-6880.
Schuldiner M, et al Exploration of the function and organization of the yeast early secretory pathway through an epistatic miniarray profile, Cell 123, 507-519 (2005)
Surmaet M. A. al, A lipid E-MAP identifies Ubx2 as a critical regulator of lipid saturation and lipid bilayer stress, Mol. Cell 51, 519-530 (2013).
Tai L M, et al. EFAD transgenic mice as a human APOE relevant preclinical model of Alzheimer's disease. J Lipid Res. 2017 September; 58(9):1733-1755
Velazquez R, et al. Maternal choline supplementation ameliorates Alzheimer's disease pathology by reducing brain homocysteine levels across multiple generations. Mol Psychiatry. 2019 Jan. 8.
Velazquez R, et al Lifelong choline supplementation ameliorates Alzheimer's disease pathology and associated cognitive deficits by attenuating microglia activation. Aging Cell. 2019 Dec.; 18(6):e13037.
Wang, L., J. Day, C. M. Roe, M. R. Brier, J. B. Thomas, T. L. Benzinger, J. C. Morris and B. M. Ances (2014). “The effect of APOE epsilon4 allele on cholinesterase inhibitors in patients with Alzheimer disease: evaluation of the feasibility of resting state functional connectivity magnetic resonance imaging.” Alzheimer Dis Assoc Disord 28(2): 122-127.
Wang Y, et al Choline Supplementation Ameliorates Behavioral Deficits and Alzheimer's Disease-Like Pathology in Transgenic APP/PS1 Mice. Mol Nutr Food Res. 2019 September; 63(18):e1801407
Zhang Y., et al, Purification and Characterization of Progenitor and Mature Human Astrocytes Reveals Transcriptional and Functional Differences with Mouse., Neuron 89, 37-53 (2016).

1. A method of treating a subject for an APOE4-related disorder comprising determining the presence or absence of an ApoE4 gene in a subject having an APOE4 related disorder and delivering to the subject an effective amount of choline supplementation if the subject has an ApoE4 gene.

2. The method of claim 1, wherein the effective amount is an effective daily dose of greater than 550 mg.

3. The method of claim 1, wherein the APOE4-related disorder is selected form the group consisting of Alzheimer's Disease (A D), cardiovascular disease, atherosclerosis, traumatic brain injury (TBI), Cerebral Amyloid Angiopathy (CAA), dementia with Lewy bodies (DLB), tauopathy, cerebrovascular disease, multiple sclerosis, and vascular dementia.

4. The method of claim 1, wherein the APOE4-related disorder further comprises APOE4-mediated lipid dysfunction.

5. The method of claim 4, wherein the APOE4-mediated lipid dysfunction comprises an accumulation of lipid droplets in microglia and/or an accumulation of lipid droplets in astrocytes.

6. A method of reducing APOE4-mediated lipid dysfunction in a subject comprising identifying a subject in need of reducing APOE4-mediated lipid dysfunction and administering to the subject an effective amount of choline supplementation, wherein APOE4-mediated lipid dysfunction comprises an accumulation of lipid droplets in microglia, an accumulation of lipid droplets in astrocytes, and/or an increase in inflammatory cytokine IL-1B in microglia cells following activation with interferon gamma.

7. A method of reducing amyloid β (Aβ) deposition in a subject comprising administering to the subject an effective amount of choline supplementation for reducing amyloid (Aβ) deposition, wherein the subject has been identified as having an ApoE4 gene and wherein the choline supplementation is administered to the subject for at least 3 months.

8. The method of claim 1, wherein the effective amount of choline supplementation is an effective amount for altering phosphatidylcholine (PC) metabolism in the subject.

9. The method of claim 8, wherein altering PC metabolism in a subject comprises increased expression of one or more of the following genes Pld3, S1pr1, or Plpp3 in astrocytes and/or increased expression of one or more of genes Lpcat2, P2ry12, Tgfbr1, Gpr34, Lyn, or Picalm in microglia relative to a control.

10. The method of claim 1, wherein the effective amount of choline supplementation is an effective amount for normalizing microglial activation in the subject.

11. The method of claim 10, wherein normalizing microglial activation comprises decreased expression of IL-1b induction following activation with interferon gamma relative to a control.

12. The method of claim 1, wherein the effective amount of choline supplementation is an effective amount for decreasing lipid droplet accumulation in the liver of the subject.

13. The method of claim 1, wherein the wherein the choline supplementation comprises a choline salt, wherein the choline salt is choline chloride, choline bitartrate or choline stearate.

14. The method of claim 1, wherein the choline supplementation is administered to a subject once a day, twice a day, or three times a day.

15. The method of claim 1, wherein the choline supplementation is administered to the subject for at least 3 months.

16. The method of claim 1, wherein the choline supplementation is administered to the subject for at least 6 months.

17. The method of claim 1, wherein the choline supplementation is administered to the subject for at least 12 months.

18. The method of claim 1, further comprising administering a cholinesterase inhibitor to the subject.
